(19) United States
(12) Patent Application Publication
(10) Pub. No.: US 2021/0032776 A1
(43) Pub. Date: Feb. 4, 2021

(54) METHOD AND SYSTEM FOR FABRICATING DNA SEQUENCING ARRAYS

(71) Applicant: CENTRILLION TECHNOLOGY HOLDINGS CORPORATION, Grand Cayman (KY)

(72) Inventors: Paul DENTINGER, Palo Alto, CA (US); Justin COSTA, Union City, CA (US); Glenn MCGALL, Palo Alto, CA (US); Filip CRNOGORAC, Redwood City, CA (US); Wei ZHOU, Saratoga, CA (US)

(21) Appl. No.: 16/982,349

(22) PCT Filed: Mar. 20, 2019
(86) PCT No.: PCT/US2019/023245

§ 371 Date: Sep. 18, 2020

Related U.S. Application Data
(60) Provisional application No. 62/646,279, filed on Mar. 21, 2018.

(51) Int. Cl. C40B 50/18; C12Q 1/6874; C12Q 1/6837

(52) U.S. Cl. CPC C40B 50/18; C12Q 1/6837; C12Q 1/6874

(57) ABSTRACT

The present disclosure relates to processes for inverting oligonucleotide probes in an in situ synthesized array. These processes can be used to reverse the orientation of probes with respect to the substrate from 3′-bound to a substrate to 5′-bound to another substrate. These processes can also be used to reduce or eliminate the presence of truncated probe sequences from an in situ synthesized array. These processes can preserve the original patterns of the synthesized oligonucleotide after the inversion. These process can be achieved via the formation of a hydrogel layer in-between a donor substrate and an acceptor substrate through a polymerization reaction forming the hydrogel layer.

### CROSS-REFERENCE

This application claims the benefit of U.S. Provisional Patent Application No. 62/646,279, filed Mar. 21, 2018, which application is entirely incorporated herein by reference.

### BACKGROUND

High-density DNA microarrays have seen extensive use in a range of applications for genomic sequence analysis, including the detection and analysis of mutations and polymorphisms (SNP genotyping), cytogenetics (copy number), nuclear proteomics, gene expression profiling, and transcriptome analysis. While many of these applications can employ direct hybridization-based methodologies for readout, the use of enzymatic readout may offer certain distinct advantages. For example, there may be much higher level of discrimination afforded by polymerase extension or ligation of the arrayed sequences, compared with detection by hybridization alone.

One method for the fabrication of very high density DNA microarrays combines in situ synthesis with photo-lithographic semiconductor manufacturing methods to provide arrays with high density DNA sequences on the substrate. The photolithographic methods can result in a population of incomplete or truncated probe sequences which accompany the probe sequences synthesized at the full desired or intended length (“full-length” probes). The presence of such truncated probe sequences can have a detrimental effect on array performance, for example, in hybridization reactions to contribute to a poor signal-to-noise ratio. However, the photo-lithographic method permits efficient oligonucleotide synthesis in the 3′ to 5′ direction with the 3′-terminus of the synthesized probe bound to the solid support (5′ up microarrays). In certain enzymatic reactions demanding enzymatic addressing of the free probe terminus, such as polymerase extension reactions or ligation reactions, a free 3′-hydroxyl is required for the enzymatic reaction to occur. The orientation of a sequence synthesized by the photo-lithographic methods is usually in the 3′→5′ direction. This leaves the 3′ end of the synthesized sequence attached to the surface and unable to participate in enzymatic reactions requiring a free 3′-hydroxyl terminus.

In contrast, oligonucleotide probes immobilized on bead arrays (e.g., Illumina) and other spotted arrays are commonly attached to their substrates via an amine or other functional groups synthetically attached to the 5′ end of the full-length, previously synthesized and purified probes. But arrays of increased complexity are difficult to be synthesized in this way. To date, 3′ up microarrays have been fabricated almost exclusively by the “top-down” microfabrication strategy in two steps: the molecules are first synthesized conventionally in the 5′ up orientation with a linker at the 5′ end of the synthesized sequences. Then the synthesized sequences are cleaved from its 3′ end, and subsequently react the 5′ end link en masse to a substrate and produce a 3′ up sequence by spotting.

### SUMMARY

It can be desirable to invert the orientation of the probes on in situ synthesized arrays, such as those fabricated with photolithography such that the probes are in 5′→3′ direction with a free 3′-hydroxyl terminus, and are in “full-length.” The present disclosure provides processes for accomplishing this molecular inversion of the orientation of the probe sequence such that probe sequences originally synthesized from the 3′ ends on a donor substrate are converted to probe sequences that are attached to an acceptor substrate via their 5′ ends to expose free 3′-hydroxyls, while maintaining the original patterns of sequences on the donor substrate on the resulting acceptor substrate. In addition, the present disclosure can also reduce or eliminate truncated oligonucleotide probes in the receptor substrate.

Current techniques for making high-resolution, photolithographic DNA microarrays may suffer from the limitation that the 3′ end of each sequence is anchored to a hard substrate and hence unavailable for many potential enzymatic reactions. The present disclosure provides a technique that can invert the entire microarray into a hydrogel. This method can preserve the spatial fidelity of the original pattern of the microarray while simultaneously removing incorrectly synthesized oligomers that are inherent to all other microarray fabrication strategies. First, a standard 5′-up microarray on a donor wafer may be synthesized, in which each oligo is anchored with a cleavable linker at the 3′ end of the oligonucleotide attached to the surface of the microarray and having an acrydite phosphoramadite at the 5′ end (hereinafter called “Acrydite”).

Acrydite or Acrydite phosphoramidite is a phosphoramidite that allows the synthesis of oligonucleotides with a methacryl group at the 5′ end, i.e., a 7-methacrylamidoheptylphosphonic acid, monoester at the 5′ end of an oligonucleotide:

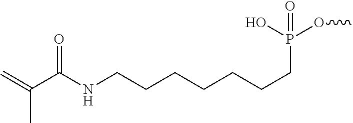

Following the completion of the synthesis of the array, an acrylamide monomer solution can be applied to the donor wafer, and an acrylamide-silanized acceptor wafer can be placed on top of the acrylamide monomer solution. As the polyacrylamide hydrogel forms between the two wafers, it covalently incorporates the Acrydite-terminated sequences into the hydrogel matrix. Finally, the oligos can be released from the donor wafer by immersion in an ammonia solution that cleaves the 3′ cleavable linkers that have been inserted between the donor wafer and the oligos, thus freeing the oligos at the 3′ end. The array can now be presented 3′ up on the surface of the gel-coated acceptor wafer. Extension reactions, restriction digests, and on gel mini-sequencing using labelled reversible terminators can demonstrate a versatile and robust platform that can easily be constructed with far more molecular complexity than traditional microarrays by endowing the system with multiple enzymatic substrates. This generation of microarrays where highly ordered, purified oligos can be inverted 3′-up, in a biocompatible soft hydrogel, and can be functional with respect to a wide variety of programmable enzymatic reactions.

This disclosure presents a solution to the problems of synthesizing high density, inverted, enzyme compatible microarrays. First, conducting a 3′→5′ synthesis (the “bottoms up” approach) for a donor wafer; then covalently anchoring the synthesized oligos into a polyacrylamide hydrogel for the sequences which are not capped and, therefore, can receive an Acrydite phosphoramidite (the “top down” approach). After cleavage at the 3′ end to separate the two wafers, the resulting array of purified oligonucleotides may be inverted with the 3′ up on the surface of a hydrogel, while retaining the spatial register of sequences from the original pattern from the “bottom up” approach methods. Beyond the advantages of being relatively inexpensive, scalable, and compatible with current machines and methods used for synthesizing microarrays, this capability may enable a new generation of high-density photolithographic arrays with unique applications that may leverage the diverse biochemistry of nucleic acid enzymes.

In one aspect, the present disclosure provides a method of inverting an oligonucleotide on a surface, comprising: (a) providing a donor substrate coupled with a plurality of chains on a first surface of the donor substrate, a chain of the plurality of chains comprising an oligonucleotide in 3′ to 5′ orientation and a first reactive group attached to a 5′ end of the oligonucleotide; (b) providing an acceptor substrate comprising a plurality of second reactive groups on a second surface of the acceptor substrate; (c) arranging the donor substrate, a reaction mixture, and the acceptor substrate in a sandwich formation such that the first surface is facing the second surface with the reaction mixture in-between the first surface and second surface; (d) subjecting the sandwich formation to an immobilization condition to form a first covalent bond between the first reactive group with the reaction mixture, and a second covalent bond between a second reactive group of the plurality of second reactive groups and the reaction mixture, thereby producing a transformed sandwich formation; and (e) releasing the donor substrate from the transformed sandwich formation, thereby providing the oligonucleotide in 5′ to 3′ orientation on the acceptor substrate.

In another aspect, the present disclosure provides a method of inverting an oligonucleotide on a surface, comprising: (a) providing a donor substrate coupled with a plurality of molecules on a first surface of the donor substrate, a member of the plurality of molecules comprising (i) a first oligonucleotide in 3′ to 5′ orientation immobilized on the first surface of the donor substrate and (ii) a first reactive group attached to a 5′ end of the first oligonucleotide; (b) providing an acceptor substrate comprising a plurality of second reactive groups immobilized on a surface of the acceptor substrate; (c) arranging the donor substrate, a reaction mixture, and the acceptor substrate in a sandwich formation such that the first surface of the donor substrate is facing the surface of the acceptor substrate and the reaction mixture is placed in-between the first surface of the donor substrate and the surface of the acceptor substrate; (d) subjecting the sandwich formation to an immobilization condition to form a first covalent bond between the first reactive group with the reaction mixture or derivative thereof, and a second covalent bond between a member of the plurality of second reactive groups and the reaction mixture or derivative thereof, thereby producing a transformed sandwich formation; (e) releasing the donor substrate from the first oligonucleotide; and (f) providing the first oligonucleotide in 5′ to 3′ orientation immobilized on the acceptor substrate via the reaction mixture or derivative thereof.

In some embodiments of aspects provided herein, the first oligonucleotide comprises a free 3′ hydroxyl group in (f). In some embodiments of aspects provided herein, the member of the plurality of molecules further comprises a universal cleavable linker in-between the first surface of the donor substrate and the first oligonucleotide in 3′ to 5′ orientation. In some embodiments of aspects provided herein, the universal cleavable linker is coupled to the first surface via a reagent of

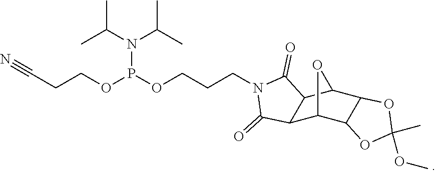 [SMILES: COC1(C)OC2C(O1)C1OC2C2C(=O)N(CCCOP(OCCC#N)N(C(C)C)C(C)C)C(=O)C12]

In some embodiments of aspects provided herein, the releasing in (e) comprises treating with a base. In some embodiments of aspects provided herein, the base comprises at least one member selected from the group consisting of NH₄OH, 1,2-diaminoethane, and methyl am. In some embodiments of aspects provided herein, the immobilization condition is a polymerization reaction. In some embodiments of aspects provided herein, the reaction mixture comprises a plurality of acrylamides for the polymerization reaction. In some embodiments of aspects provided herein, the polymerization reaction forms a polymeric gel, the polymer gel comprises the first covalent bond and the second covalent bond. In some embodiments of aspects provided herein, the first reactive group comprises a first polymerizable group. In some embodiments of aspects provided herein, the second reactive group comprises a second polymerizable group. In some embodiments of aspects provided herein, in (a) the first oligonucleotide in 3′ to 5′ orientation is full-length. In some embodiments of aspects provided herein, in (f) the first oligonucleotide in 5′ to 3′ orientation is full-length. In some embodiments of aspects provided herein, the in (e) the releasing further comprises performing a mechanical dicing process or a laser perforation process on a second surface of the donor substrate. In some embodiments of aspects provided herein, in (e) subsequent to the performing the mechanical dicing process or the laser perforation process, the releasing further comprises treating with a base. In some embodiments of aspects provided herein, the plurality of molecules form a pattern on the first surface of the donor substrate. In some embodiments of aspects provided herein, in (f) the providing comprises converting the plurality of molecules in to a plurality of inverted molecules on the surface of the acceptor substrate, and wherein the plurality of inverted molecules keep the pattern on the surface of the acceptor substrate.

In another aspect, the present disclosure provides a method of preparing an oligonucleotide array in 5′ to 3′ orientation immobilized on an acceptor surface of an acceptor substrate, comprising: (a) providing a sandwich formation, the sandwich formation comprising: (i) a donor substrate comprising a donor surface; (ii) a plurality of oligonucleotides, a 3′ end of each member of the plurality of oligonucleotides being covalently bonded to the donor surface; (iii) a middle layer covalently bonded to a 5′ end of the member of the plurality of oligonucleotides; and (iv) an acceptor substrate comprising an acceptor surface, the middle layer being covalently bonded to the acceptor surface; (b) removing the donor substrate from the plurality of the plurality of oligonucleotide; and (c) providing the oligonucleotide array in 5′ to 3′ orientation on the acceptor surface of the acceptor substrate.

In some embodiments of aspects provided herein, the method further comprises: prior to (a), forming the middle layer from a mixture of reagents in between the donor surface bonding with the plurality of oligonucleotides and the acceptor surface. In some embodiments of aspects provided herein, the forming the middle layer comprises conducting a polymerization reaction. In some embodiments of aspects provided herein, the polymerization reaction polymerizes acrylamide reagents. In some embodiments of aspects provided herein, the 3′ end of the member of the plurality of oligonucleotides is covalently bonded to a universal cleavable linker at the 3′ end of the member, the universal cleavable linker being covalently bonded to the donor surface. In some embodiments of aspects provided herein, the removing in (b) comprises breaking a bond between the universal cleavable linker and the member of the plurality of oligonucleotides. In some embodiments of aspects provided herein, the removing in (b) further comprises performing a mechanical dicing process or a laser perforation process on another surface of the donor substrate before the breaking the bond. In some embodiments of aspects provided herein, the breaking the bond comprises treating the universal cleavable linker with a basic reagent. In some embodiments of aspects provided herein, the basic reagent comprises at least one member selected from the group consisting of NH₄OH, 1,2-diaminoethane, and methyl amine. In some embodiments of aspects provided herein, after (b) the middle layer remains covalently bonded to the acceptor surface. In some embodiments of aspects provided herein, after (c) the oligonucleotide array remains in 5′ to 3′ orientation and covalently bonded to the middle layer via the 5′ end of the member of the plurality of oligonucleotides. In some embodiments of aspects provided herein, the after (c) each member of the oligonucleotide array comprises a free 3′ hydroxyl group. In some embodiments of aspects provided herein, prior to the forming the middle layer, synthesizing the plurality of oligonucleotides from the donor surface in the 3′ to 5′ orientation. In some embodiments of aspects provided herein, the middle layer is about 10 μm, 15 μm, 20 μm, 25 μm, or 30 μm thick.

In another aspect, the present disclosure provides a composition comprising: (a) a donor substrate comprising a donor surface; (b) a plurality of oligonucleotides, each member of the plurality of oligonucleotides being covalently bonded to the donor surface at a 3′ end of the member of the plurality of oligonucleotides; (c) a middle layer covalently bonded to a 5′ end of the member of the plurality of oligonucleotides; and (d) an acceptor substrate comprising an acceptor surface, the middle layer being covalently bonded to the acceptor surface.

In some embodiments of aspects provided herein, the member of the plurality of oligonucleotides is covalently bonded to a universal cleavable linker via the 3′ end of the member of the plurality of oligonucleotides. In some embodiments of aspects provided herein, the universal cleavable linker is covalently bonded to the donor surface. In some embodiments of aspects provided herein, the donor substrate is configured to be mechanically diced or laser perforated into multiple pieces. In some embodiments of aspects provided herein, the middle layer comprises polyacrylamide. In some embodiments of aspects provided herein, the donor substrate is a Silicon wafer. In some embodiments of aspects provided herein, the acceptor substrate is a quartz wafer. In some embodiments of aspects provided herein, the each member of the plurality of oligonucleotides comprises a free 3′ hydroxyl. In some embodiments of aspects provided herein, the composition is characterized in a combination of any two or more selected from the group consisting of: (i) the member of the plurality of oligonucleotides is covalently bonded to a universal cleavable linker via the 3′ end of the member of the plurality of oligonucleotides; (ii) the donor substrate is configured to be mechanically diced or laser perforated into multiple pieces; (iii) the middle layer comprises polyacrylamide; (iv) the donor substrate is a Silicon wafer; (v) the acceptor substrate is a quartz wafer; and (vi) each member of the plurality of oligonucleotides comprises a free 3′ hydroxyl. In some embodiments of aspects provided herein, the middle layer is about 10 μm, 15 μm, 20 μm, 25 μm, or 30 μm thick

In another aspect, the present disclosure provides a composition comprising: (a) a substrate comprising a surface; (b) a middle layer comprising a first surface and a second surface, the first surface being proximal to the surface of the substrate and the second surface being distal to the surface of the substrate, the first surface covalently bonded to the surface of the substrate; and (c) a plurality of oligonucleotides covalently bonded to the second surface of the middle layer via 5′ ends of the plurality of oligonucleotides.

In some embodiments of aspects provided herein, the 5′ ends of the plurality of oligonucleotides bonded to the second surface via carbon-carbon bonds. In some embodiments of aspects provided herein, the substrate is quartz. In some embodiments of aspects provided herein, the middle layer comprises polyacrylamide. In some embodiments of aspects provided herein, the surface of the substrate is bonded to the first surface via carbon-carbon-bonds. In some embodiments of aspects provided herein, each member of the plurality of oligonucleotides comprises a free 3′ hydroxyl. In some embodiments of aspects provided herein, the composition is characterized in a combination of any two or more selected from the group consisting of: (i) the 5′ ends of the plurality of oligonucleotides bonded to the second surface via carbon-carbon bonds; (ii) the substrate is quartz; (iii) the middle layer comprises polyacrylamide; (iv) surface of the substrate is bonded to the first surface via carbon-carbon-bonds; and (v) each member of the plurality of oligonucleotides comprises a free 3′ hydroxyl. In some embodiments of aspects provided herein, the middle layer is about 10 μm, 15 μm, 20 μm, 25 μm, or 30 μm thick.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1F show a schematic process for inverting a probe by the disclosed microarray inversion method into a hydrogel. FIG. 1A depicts that oligos can be prepared 5′ up on a donor substrate modified by an oligonucleotide sequence comprising a universal cleavable linker (UCL) and a 5′ Acrydite. FIG. 1B shows that an acrylamide coated acceptor substrate can be prepared. FIG. 1C depicts that an acrylamide solution can be poured onto the donor substrate while the acceptor can be inverted and placed on top of the poured acrylamide solution. FIG. 1D shows that the acceptor wafer can be either mechanically diced or perforated by laser. FIG. 1E depicts that after exposure to concentrated ammonia (e.g., 28-33% ammonia in water, also called ammonium hydroxide), for example, for about 18 hours with agitation, the wafers can be separate. FIG. 1F shows that the transferred array can be 3′ up on the acceptor wafer.

FIGS. 2A-2C demonstrate that patterned AM1 DNA and be transferred into a polyacrylamide hydrogel. FIG. 2A shows a fluorescently labeled probe was hybridized to the synthesized oligos on the gel of the acceptor substrate after using a resolution test pattern and using DMT chemistry on a 2 in×3 in substrate.

FIG. 2B shows a magnified image of a section (an inset) of the fluorescence imaging shown in FIG. 2A of the fluorescently labeled probe hybridized to the synthesized oligos on the gel. This magnified inset from FIG. 2A may demonstrate the transfer fidelity and high resolution of the pattern.

FIG. 2C shows fluorescence imaging of the fluorescently labeled probe hybridized to the synthesized oligos on the gel of the acceptor substrate. The oligos on the donor substrate of a 6 in wafer were synthesized using the photoamidite method. FIG. 2C displays 3 μm (left side) and 8 μm (right side) square features on a 6 in acceptor substrate (wafer), which may demonstrate the scalability of the process.

FIG. 3A shows fluorescent images of Cy3 labelled extended nucleotide from Taq polymerase-catalyzed extension reactions using labelled T only while in the presence of all 4 bases, 3 μm square features.

FIG. 3B shows fluorescent images of Cy3 labelled extended nucleotide from Hero polymerase extension of labelled A in the presence of all 4 labelled bases.

FIG. 4 shows a fluorescent image of transferred oligos with resolution defined by photoresist process demonstrating 1 μm line and space patterns, approximately the lithographic limit of the imaging apparatus used.

FIG. 5A shows fluorescent microscopy of sequencing by synthesis for the first base on an inverted 3′ up oligo array prepared by using the disclosed method with reversible terminators. FIG. 5B shows the sequences of the template and the growing chain such that there is a direct match for the first base (cytosine at the 3′ end of the immobilized oligonucleotide).

FIG. 5C shows fluorescent microscopy of another sequencing by synthesis for the second base on an inverted 3′ up oligo array prepared by using the disclosed method with reversible terminators. FIG. 5D shows the sequences of the template and the growing chain such that there is a direct match for the second base (adenine at the 3′ end of the immobilized oligonucleotide) after cleavage of the blocking group on the first added reversible terminator and a second round of extension.

FIG. 6A shows an example phosphoramidite reagent to make a universal cleavable linker. FIG. 6B shows another example phosphoramidite reagent to make a universal cleavable linker. FIG. 6C shows still another example phosphoramidite reagent to make a universal cleavable linker.

FIGS. 7A-7D are schematic diagrams showing the transferred oligos are 3′ up on acceptor hydrogel surface and are enzymatically functional. FIG. 7A shows fluorescence imaging after the inverted 3′ up oligo array was hybridized with a template oligo and extended by Klenow DNA polymerase with all 4 unlabeled bases. FIG. 7B shows fluorescence imaging after the extension reactions of FIG. 7A when the template oligo was stripped away with 0.2 M NaOH, and a Cy3 labelled probe targeting the newly synthesized Mosaic End sequence was added. FIG. 7C shows fluorescence imaging after exposure of the 3′ up oligos on the array of FIG. 7B to the restriction enzyme Ecor1 to digest part of the extended oligos on the array. FIG. 7D shows fluorescence imaging of the 3′ up oligos on the array of FIG. 7C when a labelled probe with the complement to AM1 is added, showing that the patterned DNA from the original array in FIG. 7B is intact after the restriction enzyme treatment in FIG. 7C.

### DETAILED DESCRIPTION

The present disclosure provides processes for the inversion of in situ synthesized oligonucleotide probes. The processes disclosed herein can also reduce or eliminate truncated oligonucleotide probes, which do not contain the full-length of the synthesized oligonucleotide sequence, while preserving full-length oligonucleotide probes. For example, full-length oligonucleotides can be immobilized to the acceptor substrate prior to release of the 3′ ends from the donor substrate, while non-full-length oligonucleotides cannot be immobilized to the acceptor substrate, and therefore can be removed upon release of the 3′ ends after the immobilization step.

The term “oligonucleotide” as used herein generally refers to a nucleotide chain. In some cases, an oligonucleotide is less than 200 residues long, e.g., between 15 and 100 nucleotides long. The oligonucleotide can comprise at least or about 1, 2, 3, 4, 5, 6, 7, 8, 9, 10, 15, 20, 25, 30, 35, 40, 45, or 50 bases. The oligonucleotides can be from about 3 to about 5 bases, from about 1 to about 50 bases, from about 8 to about 12 bases, from about 15 to about 25 bases, from about 25 to about 35 bases, from about 35 to about 45 bases, or from about 45 to about 55 bases. The oligonucleotide (also referred to as “oligo”) can be any type of oligonucleotide (e.g., a primer). Oligonucleotides can comprise natural nucleotides, non-natural nucleotides, or combinations thereof.

The term “about” as used herein generally refers to +/−10%, 9%, 8%, 7%, 6%, 5%, 4%, 3%, 2%, or 1% of the designated amount.

As used herein, the term “3′→5′ direction” or “3′ to 5′ orientation” generally means that the orientation of a nucleic acid sequence has its 3′ end of the nucleic acid sequence attached to/immobilized on the surface of a substrate. As used herein another term “5′ up” generally describes the 3′-5′ orientation as well.

As used herein, the term “5′→3′ direction” or “5′ to 3′ orientation” generally means that the orientation of a nucleic acid sequence has its 5′ end of the nucleic acid sequence attached to/immobilized on the surface of a substrate. As used herein another term “3′ up” generally describes the 5′-3′ orientation as well.

The term “immobilization” as used herein generally refers to forming a covalent bond between two reactive groups. For example, polymerization of reactive groups is a form of immobilization. A Carbon to Carbon covalent bond formation is an example of immobilization. Genetic information can be utilized in a myriad of ways with the advent of rapid genome sequencing and large genome databases. One of such applications is oligonucleotide arrays. The general structure of an oligonucleotide array, or commonly referred to as a DNA microarray or DNA array or a DNA chip, is a well-defined array of spots or addressable locations on a surface. Each spot can contain a layer of relatively short strands of DNA called “probe” or “capture probe” (e.g., Schena, ed., “DNA Microarrays A Practical Approach,” Oxford University Press; Marshall et al. (1998) Nat. Biotechnol. 16:27-31; each incorporated herein by reference). There are at least two technologies for generating arrays. One is based on photolithography (e.g. Affymetrix) while the other is based on robot-controlled ink jet (spotbot) technology (e.g., Arrayit.com). Other methods for generating microarrays are known and any such known method may be used herein.

Generally, an oligonucleotide (probe or capture probe) placed within a given spot in the array can be selected to bind at least a portion of a nucleic acid or complimentary nucleic acid of a target nucleic acid. An aqueous sample can be placed in contact with the array under the appropriate hybridization conditions. The array then can be washed thoroughly to remove all non-specific adsorbed species. In order to determine whether or not the target sequence has been captured, the array can be “developed” by adding, for example, a fluorescently labeled oligonucleotide sequence that is complimentary to an unoccupied portion of the target sequence. The microarray then can be “read” using a microarray reader or scanner, which outputs an image of the array. Spots that exhibit strong fluorescence can be positive for that particular target sequence.

A probe can comprise biological materials deposited so as to create spotted arrays. A probe can comprise materials synthesized, deposited, or positioned to form arrays according to other technologies. Thus, microarrays formed in accordance with any of these technologies may be referred to generally and collectively hereafter for convenience as “probe arrays.” The term “probe” is not limited to probes immobilized in array format. Rather, the functions and methods described herein can also be employed with respect to other parallel assay devices. For example, these functions and methods may be applied when probes are immobilized on or in beads, optical fibers, or other substrates or media.

In methods and systems of the present disclosure, probes can be attached to a solid substrate. Probes can be bound to a substrate directly or via a linker. Linkers can comprise, for example, amino acids, polypeptides, nucleotides, oligonucleotides, or other organic molecules that do not interfere with the functions of probes.

The solid substrate can be biological, non-biological, organic, inorganic, or a combination of any of these. The substrate can exist as one or more particles, strands, precipitates, gels, sheets, tubing, spheres, containers, capillaries, pads, slices, films, plates, slides, or semiconductor integrated chips, for example. The solid substrate can be flat or can take on alternative surface configurations. For example, the solid substrate can contain raised or depressed regions on which synthesis or deposition takes place. In some examples, the solid substrate can be chosen to provide appropriate light-absorbing characteristics. For example, the substrate can be a polymerized Langmuir Blodgett film, functionalized glass (e.g., controlled pore glass), silica, titanium oxide, aluminum oxide, indium tin oxide (ITO), Si, Ge, GaAs, GaP, SiO₂, SiN₄, modified silicon, the top dielectric layer of a semiconductor integrated circuit (IC) chip, or any one of a variety of gels or polymers such as (poly)tetrafluoroethylene, (poly)vinylidenedifluoride, polystyrene, polycarbonate, polydimethylsiloxane (PDMS), polymethylmethacrylate (PMMA), polycyclicolefins, or combinations thereof.

Solid substrates can comprise polymer coatings or gels, such as a polyacrylamide gel or a PDMS gel. Gels and coatings can additionally comprise components to modify their physicochemical properties, for example, hydrophobicity. For example, a polyacrylamide gel or coating can comprise modified acrylamide monomers in its polymer structure such as ethoxylated acrylamide monomers, phosphorylcholine acrylamide monomers, betaine acrylamide monomers, and combinations thereof.

As used herein, the term “middle layer” generally refers to a hydrogel or gel or a polymerized layer that is bonded with a substrate, for example, the acceptor substrate, on one of its surfaces and bonded with 5′ ends of a plurality of oligonucleotide one another of its surfaces. The middle layer is in-between two substrates. The middle layer remains intact after the removal of one of the substrates, for example, the donor substrate. The 5′ ends of the plurality of oligonucleotides remain covalently bonded with the middle layer after the removal of one of the substrates, for example, the donor substrates.

As used herein, the term “hydrogel” generally refers to a gel in which the swelling agent is water. The term “gel” refers to a non-fluid colloidal network or polymer network that is expanded through its volume by a fluid. The term “swelling agent” is a fluid used to swell a gel or network. For example, water can be a swelling agent for a hydrogel. The hydrogels of the present disclosure may be prepared by polymerization of one or more acrylamide-functionalized monomers. For example, an acrylamide tail can be bonded to the 5′ ends of the plurality of oligonucleotides. An acrylamide tail can also be bonded to the surface of a substrate, for example, an acceptor substrate. Then when a solution containing acrylamide monomers is poured over one surface of a substrate bonded with acrylamide tails, another surface that is bonded with acrylamide tails can be stacked on top of the poured solution. Then the poured solution can be subject to polymerization of acrylamide monomers and the acrylamide tails such that a middle layer can be formed. In some cases, the hydrogel of the present disclosure comprises polyacrylamides. In some cases, the hydrogel of the present disclosure comprises crossed lined polyacrylamides. In some cases, the hydrogel of the present disclosure comprises about 0.5%, 1%, 2%, 3%, 4%, 5%, 6%, 7%, 8%, 9%, 10%, 11%, 12%, 13%, 14%, 15%, 16%, 17%, 18%, 19%, or 20% of polyacrylamides in weight. In some cases, the hydrogel can be obtainable by combining acrylamide and methylene bis-acrylamide. The polymerization reaction can be radical initiated by an initiator. The hydrogel can be obtained by combining acrylamide and methylene bis-acrylamide is in a molar ratio of 150:1 to 1000:1 in the presence of a radical initiator. Methylene bis-acrylamide can provide cross-linking between polymer chains and the molar ratio may be varied to provide various cross-linking densities of the hydrogel. The conditions for obtaining the hydrogel may be modified. Ammonium persulfate (AMPS) can be used as an initiator for the polymerization.

DNA microarrays can be fabricated using spatially-directed in situ synthesis or immobilization of pre-synthesized oligonucleotides. In both cases, synthesis of the oligonucleotides typically can proceed with the addition of monomers in the 3′-to-5′ direction, using standard 3′-phosphoramidite reagents and solid-phase synthesis protocols (e.g., M. Egli, et al., ed. “Current Protocols in Nucleic Acid Chemistry,” John Wiley & Sons). The main impurities are truncated, partial-length sequences resulting from incomplete monomer coupling and, to a lesser extent, depurination reactions.

On the one hand, fabricating arrays of pre-synthesized oligonucleotide probes typically can involve covalent attachment of the oligonucleotides to a substrate through the 5′-terminus, via a reactive modifier which is added to the end when the oligonucleotides are synthesized on high-throughput synthesizers (see S. J. Beaucage, et al., Curr. Med. Chem. 2001, 8, 1213-44). This ensures that the probes which are attached to the support can be primarily full-length sequences, since truncated sequences can be capped and rendered non-reactive during synthesis (Brown T and Brown T, Jr. (2005-2015) Solid-phase oligonucleotide synthesis. [Online] Southampton, UK, ATDBio. <http://www.atdbio.com/content/17/Solid-phase-oligonucleotide-synthesis>[Accessed Aug. 9, 2016]).

An advantage of the present disclosure can be that the 3′-hydroxyl group of the oligonucleotide probe is “distal” to the substrate, and can be freely available for enzymatic reactions, such as template-directed polymerase-catalyzed chain extension and ligation; and this character can be exploited to carry out very sensitive and specific assays for detecting and quantitating genetic polymorphisms (K. Lindroos, et al., Nucleic Acids Res. 2001, 29, e69; Gunderson K L, et al., Nature Genetics 2005, 37, 549-54).

On the other hand, DNA microarrays can also be fabricated using in situ synthesis of sequences directly on the support. In this case, sequences can be “printed” in a highly parallel fashion by spatially-directing the synthesis using inkjet (T. R. Hughes, et al., Nature Biotechnol 2001, 19, 342-7; C. Lausted, et al., Genome Biol 2004, 5, R58), photolithographic technologies (A. C. Pease, et al., Proc Natl Acad Sci USA 1994, 91, 5022-6; G. McGall, et al., Proc Natl Acad Sci USA 1996; 93:13555-60; S. Singh-Gasson, et al., Nature Biotechnol 1999, 17, 974-8;), or electrochemical techniques (PLoS ONE 2006, 1, e34; B. Y. Chow, et al., Proc Natl Acad Sci USA 2009, 106, 15219-24). Here too, synthesis proceeds in the 3′ to 5′ direction (solid-phase oligonucleotide synthesis in the 5′-to-3′ direction, while feasible, is much less efficient and economical, providing lower yields and product purity). However, the resulting probes can be attached to the substrate at the 3′-terminus, and any truncated sequence impurities which arise during the synthesis remain on the support, which may be a particular issue in the case of photolithographic synthesis (J. Forman, et al., Molecular Modeling of Nucleic Acids, Chapter 13, p. 221, American Chemical Society (1998) and G. McGall, et al., J. Am. Chem. Soc. 119:5081-5090 (1997)). As a result, polymerase-based extension assays normally are not feasible using arrays made this way and with this direction (5′ to 3′).

Despite the above limitation, photolithographic synthesis is a highly attractive means of fabricating very high-density DNA arrays, as it is capable of exceeding 10 million arrayed sequences per cm² (A. R. Pawloski, et al., J Vac Sci Technol B 2007, 25, 2537-46), and is highly scalable in a manufacturing setting. Thus, it is desirable to develop an effective method of inverting the sequences on such probe arrays.

For example, modern high-density DNA microarrays may combine in situ synthesis with photo-lithographic semiconductor manufacturing methods to provide arrays with densities on the order of 10⁷ discrete sequence features per cm² or greater (McGall, G. H.; Christians, F. C. High-Density Genechip Oligonucleotide Probe Arrays. Adv. Biochem. Eng. Biotechnol. 2002, 77, 21-42). This method may employ a type of “bottoms up” fabrication strategy where each base is added sequentially upon exposure through a mask. Microarrays fabricated in this way may have seen extensive use in a range of applications for molecular biology that include SNP genotyping, cytogenetics, nuclear proteomics, and massively parallel analysis of the transcriptome. Yet the versatility of microarrays may belie the fact that virtually all of their associated assays are limited to detecting hybridization events by fluorescence. There may be an extraordinary variety of enzymes utilize DNA as a substrate such that if one may invert the orientation of nucleic acid probes made by the photo-lithographic semiconductor manufacturing methods within a gel, one may endow the microarrays with new capabilities and enzymatic functionalities.

There may be several structurally inherent limitations to previously reported microarrays that may largely restrict their use with enzymes. First, the array substrate may be a hard surface such as quartz or silicon which can negatively impact the activity of enzymes with oligonucleotides in proximity to the surface; even when hydrophilic linking groups are included “in-line” to lift the oligos into a more enzymatically cooperative environment. See Shchepinov, M. S. et al., Steric Factors Influencing Hybridisation of Nucleic Acids to Oligonucleotide Arrays. Nucleic Acids Res. 1997, 25 (6), 1155-1161. Second, there may be a limit to the length of oligos on microarrays that can be fabricated by sequential base addition due to the inefficient coupling yield of phosphoramidite chemistry. Even though the value of long, pure sequences may have been established, the coupling inefficiency during synthesis results in many truncated oligomer products intermixed with the full-length sequences, and no straightforward method has been available to selectively remove them. See LeProust, E. M., et al.; Synthesis of High-Quality Libraries of Long (150 mer) Oligonucleotides by a Novel Depurination Controlled Process. Nucleic Acids Res. 2010, 38 (8), 2522-2540.

As discussed above, one constraint with previously reported photolithographic microarrays is the directional orientation of sequences, which may be synthesized using phosphoramidite chemistry in the 3′→5′ direction. This may leave the 3′ end of the array sequences attached to the surface (3′ down) and unable to participate in enzymatic reactions requiring a free 3′-hydroxyl group. To date, 3′ up microarrays have been fabricated using a “top-down” approach where the molecules may be synthesized in a 5′ up orientation with a linker at the 5′ end, then cleaved and using the cleaved oligos to react to a substrate to produce the oligo products that are 3′ up either by spotting or on beads. However, arrays manufactured in this manner may lose the scale and precision achieved by the photolithographic—a “bottoms up” fabrication strategy. One might consider using the photoamidite method of direct 5′→3′ synthesis to realize a 3′ up array. See Albert, T. J.; Norton, J., et al.; Light-Directed 5′-->3′ Synthesis of Complex Oligonucleotide Microarrays. Nucleic Acids Res. 2003, 31 (7), e35. However, lower yields of photoamidite vs. DMT-chemistry may make synthesizing a long pure oligos sequence of the correct sequence unachievable by this method.

The plurality of probes can be located in one or more addressable regions (spots, locations, etc.) on a solid substrate, herein generally referred to as “pixels.” In some cases, a solid substrate comprises at least about 2, 3, 4, 5, 6, or 7-10, 10-50, 50-100, 100-500, 500-1,000, 1,000-5,000, 5,000-10,000, 10,000-50,000, 50,000-100,000, 100,000-500,000, 500,000-1,000,000 or over 1,000,000 pixels with probes. In some cases, a solid substrate comprises at most about 2, 3, 4, 5, 6, or 7-10, 10-50, 50-100, 100-500, 500-1,000, 1,000-5,000, 5,000-10,000, 10,000-50,000, 50,000-100,000, 100,000-500,000, 500,000-1,000,000 or over 1,000,000 pixels with probes. In some cases, a solid substrate comprises about 2, 3, 4, 5, 6, or 7-10, 10-50, 50-100, 100-500, 500-1,000, 1,000-5,000, 5,000-10,000, 10,000-50,000, 50,000-100,000, 100,000-500,000, 500,000-1,000,000 or over 1,000,000 pixels with probes.

In some cases it is useful to have pixels which do not contain probes. Such pixels can act as control spots in order to increase the quality of the measurement, for example, by using binding to the spot to estimate and correct for non-specific binding. In some cases, the density of the probes can be controlled to either facilitate the attachment of the probes or enhance the ensuing detection by the probes.

In some examples, it is useful to have redundant pixels which have identical probe sequences to another pixel but physically may not be adjacent or in proximity to the other pixel. The data acquired by such probe arrays may be less susceptible to fabrication non-idealities and measurement errors.

In some cases, labels are attached to the probes within the pixels, in addition to the labels that are incorporated into the targets. In such systems, captured targets can result in two labels coming into intimate proximity with each other in the pixel. As discussed before, interactions between specific labels can create unique detectable signals. For example, when the labels on the target and probe, respectively, are fluorescent donor and acceptor moieties that can participate in a fluorescent resonance energy transfer (FRET) phenomenon, FRET signal enhancement or signal quenching can be detected.

### Synthesis of Inverted Oligonucleotides

In some cases, high density oligonucleotide features and arrays can be fabricated in a method disclosed herein. For example, oligonucleotide synthesis in 3′→5′ direction protocols, for example, the phosphoramidite chemistry, can be utilized to produce sequences in the 3′→5′ direction on a donor substrate, wherein the final 5′-end unit of a “full-length” sequence can comprise a reactive group for further chemical reactions. Then only the sequences which are “full-length” on the donor substrate are transferred en masse to a polyacrylamide hydrogel-coated receptor substrate, resulting in the “full-length” sequences immobilized in the polyacrylamide hydrogel on the acceptor substrate with inversion of the probe orientation (5′ attachment) and complete retention of the spatial arrangement of sequences from the originating array on the donor substrate. Possible application of such DNA sequencing arrays can be used as extension-based genotyping arrays and minisequencing by synthesis. The capability of producing such high density DNA sequencing arrays will enable a new generation of high-density photolithographic arrays with unique functionalities allowing the development of novel applications to leverage the highly specific biochemistry of DNA enzymes.

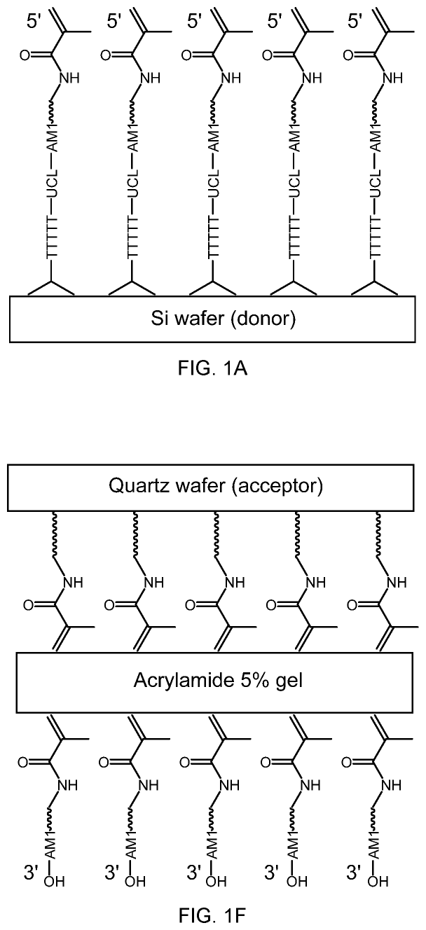

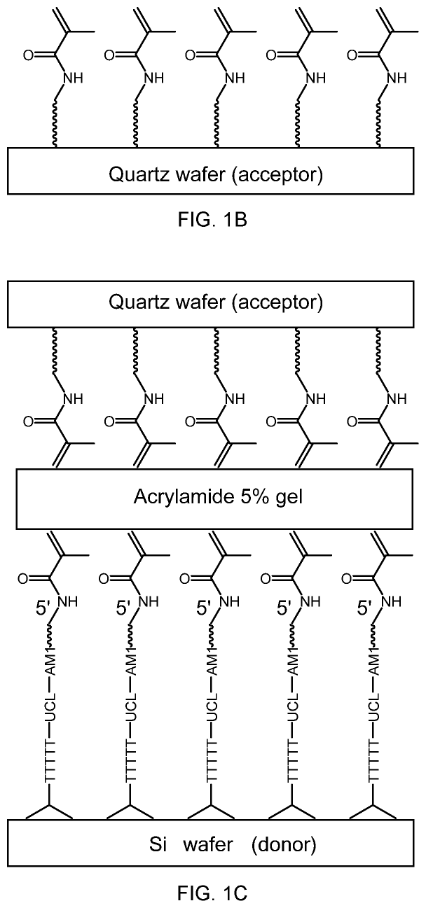

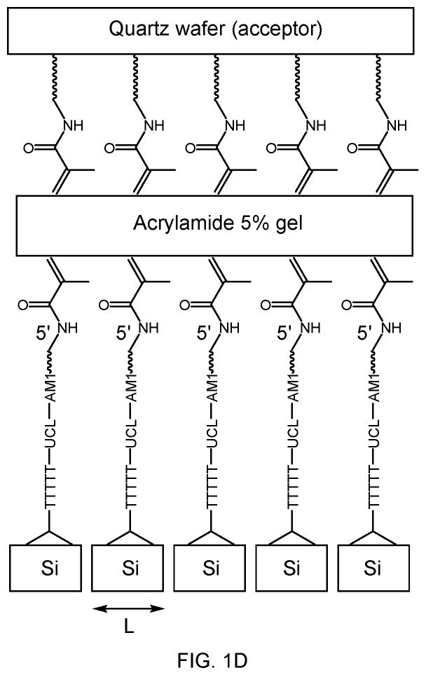

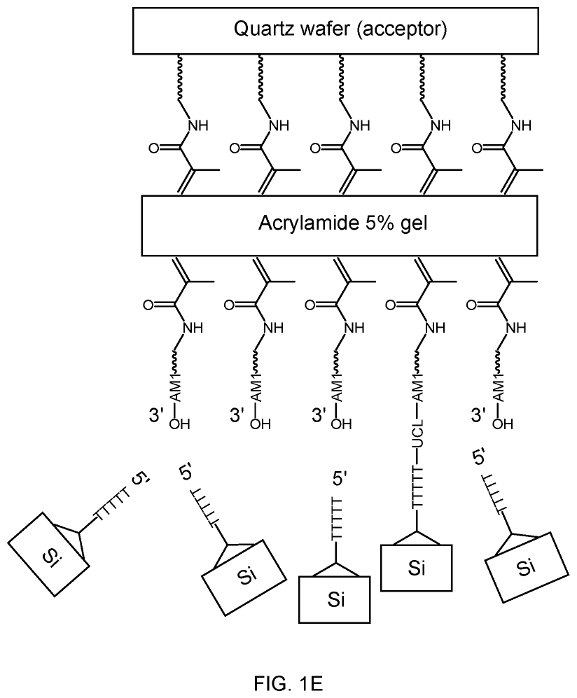

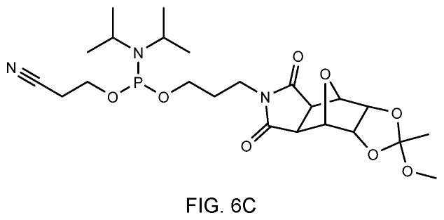

FIGS. 1A-1F show an example scheme of the method. Firstly (FIG. 1A), a cleavable silane, for example, 2-hydroxyethyl 3-(methyl(3-(trimethoxysilyl)propyl)amino)propanoate, can be applied to a silicon substrate (shown as Si wafer (donor)), and poly-(T) sequences can be synthesized using DMT-blocking chemistry with a universal cleavable linker (e.g., a phosphoramidite shown in FIG. 6A, 6B, or 6C) incorporated. This universal phosphoramidite reagent can be available at AM Chemicals, Oceanside, Calif. Variable region oligonucleotides can be applied using photolytic blocking chemistry in 3′→5′ fashion as is for microarrays and described elsewhere to create patterned structures with known DNA sequences at specific locations (Glenn McGall, “The Efficiency of Light-Directed Synthesis of DNA Arrays on Glass Substrates,” JACS, 119 (22): 5081-5090, (1997)) to make probe sequences (denoted as AM1 in FIG. 1). In some cases, the last amidite of the synthesis can be patterned with a photoamidite followed by the addition of the Acrydite moiety (FIG. 1A). In some cases, the last amidite added to the amidite pattern on the 5′-end of the AM1 sequence in the synthesis can be Acrydite. In some cases for demonstrating the effects of high resolution, photoresist can be used to pattern DMT prior to acrylamido addition.

For the acceptor wafer (FIG. 1B), the surface of the acceptor wafer can be modified to include acrylamide groups by silanization. In some cases, an acrylamide pre-gel polymerization solution can be prepared in water and quickly applied to a first substrate (either the acceptor wafer or the donor wafer), and the second substrate is immediately inverted onto the solution on the first substrate. In some cases, an acrylamide monomer solution prepared in water can be applied to the donor wafer while the acceptor wafer can be immediately inverted and placed on top forming the sandwich (FIG. 1C). Not to be limited by any working theory disclosed herein, the capillary forces may spread the polymerization solution, i.e., the monomers solution, evenly to cover either an individual die or the wafers (e.g., wafer with 6 inch in diameter), creating a “sandwich” configuration shown in the FIG. 1C. In some cases, polymerization can occur over 60 min, covalently connecting the two wafers through the hydrogel thus formed. In some cases, polymerization conditions can be allowed for from about 20 to about 60 min binding the two substrates (e.g., donor and acceptor wafers, in FIG. 1C).

In the case of small (about 1 cm pieces), the substrates can be submerged in concentrated ammonia to cleave the UCL, where 10-18 hours may be necessary for the two wafer pieces to separate. For larger substrates (e.g., six inch wafers) another step may be optionally added or required, for example, subjecting the sandwiched substrates to either a mechanical dicing process or a laser perforation process along the dicing streets (FIGS. 1D, 1E), to separate the two substrate by the treatment with a base, such as ammonia. For example, the laser perforation method can focus laser energy onto a minute area of the substrate for a very short time, thereby subliming and evaporating the solid. FIG. 1D shows that the acceptor wafer can be either mechanically diced or perforated by laser. The dimensions of the diced or perforated pieces may be about 5 mm, 6 mm, 7 mm, 8 mm, 9 mm, 10 mm, 11 mm, 12 mm, 13 mm, 14 mm, or 15 mm in length or diameter. FIG. 1E depicts that after exposing to concentrated ammonia (e.g., 28-33% ammonia in water, also called ammonium hydroxide), for example, for about 18 hours with agitation, the diced or perforated donor substrate (e.g., Si wafers) can be removed and released from the acceptor substrate (e.g., quartz wafer). For smaller donor substrates that have not been diced or perforated, similar treatment with concentrated ammonia can remove and release the acceptor substrate as well by basic hydrolysis of the UCL moieties.

In some cases, when mechanical dicing is performed, the sandwiched wafers can be mounted on dicing tape (DU-300 from Nitto, Teaneck, N.J.), and the top wafer (donor wafer) can be diced into 7.5 mm×7.5 mm squares (chips). The tool used was DISCO 2H6T dicing saw, spindle speed of about 26,000 rpm, feed rate of about 1 mm/s, using a resin bonded diamond blade (Thermocarbon, Casselberry, Fla.) of about 0.3 mm width. The cut depth was about 0.715 mm which can cut through the top wafer (donor wafer) and just touch the bottom wafer (acceptor wafer).

In some cases, laser perforation can be performed by or using the protocols from Potomac Photonics, Inc. (Baltimore Md.). A 6 in sandwiched wafers with the top wafer (donor wafer, silicon wafer) facing the laser can be perforated at 1.75 mm intervals defining 7.5 mm×7.5 mm chips. The hole diameter as estimated can be at about 0.2 mm. The bottom wafer (acceptor wafer) in this approach can be of quartz material which is transparent to the laser light (Nd:YAG, wavelength 1064 nm), such that the perforation process an stop at the quartz wafer interface (after drilling a hole through the donor silicon wafer). The process can take about 45 mins to make about 6000 holes covering the surface of the whole wafer.

After releasing the donor substrate, the donor wafer can be subsequently submerged in ammonia for at least 3 hours and/or in a 1:1 ethylenediamine (EDA):water mixture for from about one to about three hours to complete the deprotection and ensure that the universal cleavable linker (UCL) is cleaved to reveal the 3′ hydroxyl (i.e., cleavage of the UCL to reveal the 3′ hydroxyl group on the DNA sequences). The wafer can then be rinsed with water, then 4× saline-sodium citrate (SSC) buffer, ready for further analysis and/or reactions (FIG. 1F).

The following test can show that the as-synthesized oligos are transferred with high fidelity to the gel coated acceptor wafer. As an example to demonstrate that using the above-described methods the oligos or DNA sequences on the donor substrate can be transferred to the gel on the acceptor substrate with good fidelity, a 20-mer (5′TACGATTCAGCCGATACAGC3′, AM1) can be synthesized using DMT chemistry on a 2 in×3 in donor substrate. Next, four DMT-thymine residue can be added to the 5′ end of the 20-mer, and then a thymine phosphoramidite with a photoactivatable blocking group (photo-T) can be added. Finally, the photo-T can be selectively reacted by UV exposure through a resolution test pattern mask (e.g., a Centrillion resolution test pattern or RTP), and the acrylamido phosphoramidite (ACRYDITE™, Glen Research, Sterling, Va.) can then be added to the oligonucleotide via the exposed hydroxyl group. In some cases, the acrylamido phosphoramidite (ACRYDITE™) can be added only in the exposed, deblocked regions. In other cases, the acrylamido phosphoramidite can be added to the entire surface of the treated donor substrate. The areas exposed through the mask (deblocked regions) can react with the ACRYDITE™ phosphoramidite, even when the ACRYDITE™ may be in contact with the entire wafer surface. Then the sandwich assembly can be shaken in ammonia for 24 hours to cleave the universal cleavable linker (UCL), thus releasing the oligos from the donor wafer into the hydrogel.

As used herein a photo nucleoside phosphoramidite or photoamidite, including photo-T, can be a nucleoside analog/reagent that comprises (i) a photo-sensitive protecting group on the nucleoside, for example, on the 5′ hydroxyl group, and (ii) a phosphoramidite moiety on the 3′ hydroxyl, as shown below:

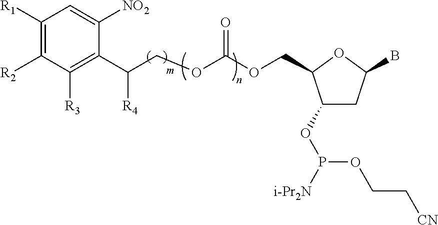

wherein:

each of R₁, R₂ and R₃ is independently H, alkyl, alkoxy, or aryl, or any two of R₁, R₂ and R₃ together with the atoms bonded thereto form a fused ring with the benzene ring bearing the nitro group;

R₄ is H, alkyl or aryl;

m is 0 or 1;

n is 0 or 1;

B is protected nucleic acid heterocyclic bases: A^{pg}, C^{pg}, G^{pg}, T, U;

A is adenine;

C is cytosine;

G is guanine;

T is thymine;

U is uracil; and

pg is independently a protecting group or protecting groups on exocyclic nitrogen atoms of heterocyclic bases A, C, G, T or U.

The UCL can be a molecule that is non-reactive during the oligonucleotide synthesis, but can be reactive after the completion of the oligonucleotide synthesis to release a free 3′-OH termini. Choices for the universal cleavable linker (UCL) can include, but are not limited to, molecules shown in FIG. 6A, FIG. 6B, and FIG. 6C. Multiple UCLs can be inserted in-between the poly-T sequence and the synthesized 3′ to 5′ oriented oligos.

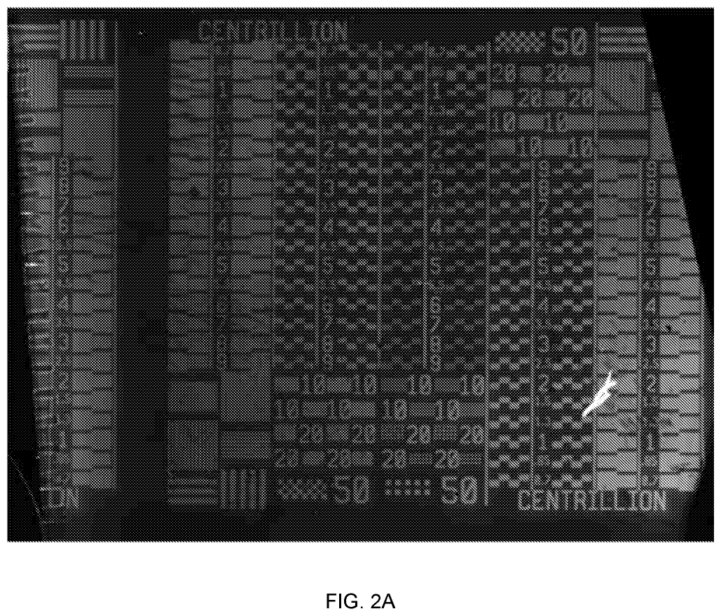

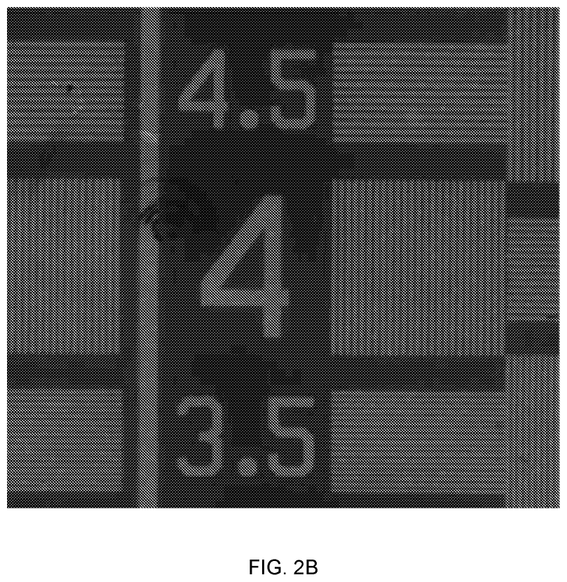

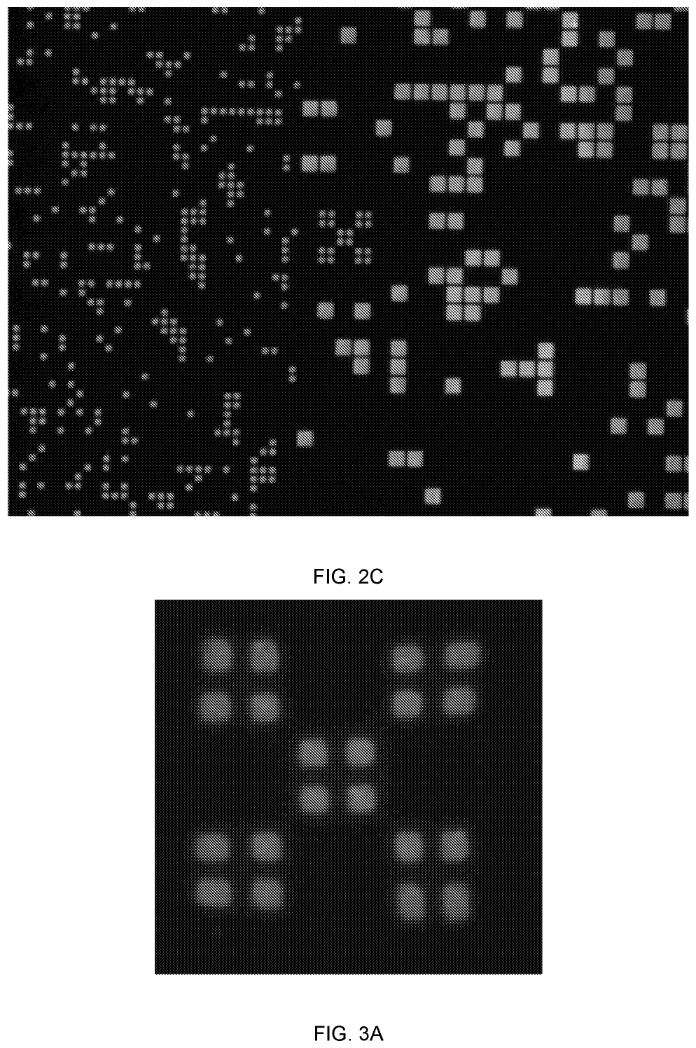

To verify successful transfer of the oligos into the gel, a fluorescently tagged compliment to the AM1 sequence was hybridized and imaged at 10× magnification (FIG. 2A). The resolution test pattern mask has a 5.5 mm field, with 500 μm between fields. Feature fidelity and hybridization signal intensity were maintained across the 7.5 mm piece as shown. FIG. 2B shows an inset from 2A, illustrating that the achieved spatial resolution after transfer is high, with a 3-4 μm line and space pattern as shown. Finally, to demonstrate that the process is compatible with all microarray fabrication requirements, a full 6-inch wafer was synthesized with the cleavable silane, two UCLs and the AM1 sequence via the photoamidite method (FIG. 2C). Laser perforation along the dicing streets of the donor wafer may facilitate mass transfer of the ammonia to areas of the wafer modified by the cleavable silane. After gel transfer, 3- and 8-micron features were readily identified, which may demonstrate that the whole process can be scaled up. These results demonstrate that highly ordered arrays of oligos can be transferred into a hydrogel coated acceptor wafer at relevant die geometries while maintaining high spatial pattern fidelity. Diffusion of ammonia through the polymerized hydrogel is clearly sufficient to enable chemical cleavage of the moieties synthesized below the photo-defined sequence, and the chemistry utilized is compatible with commercial microarray fabrication techniques.

The probes on the gel can be hybridized with the complement of the as-synthesized AM1 tagged with Cy3 on the 5′ end (QCAM1, IDT, Coralville, Iowa) and imaged, at 10× magnification and are shown in FIG. 2A. The Centrillion RTP is 5.5 mm field, with 0.5 mm between fields. As shown in FIG. 2A, the feature fidelity and signal can be maintained across the ˜7.5 mm piece shown.

In some cases, DMT chemistry may not be compatible with photolithographically-based microarray probe synthesis because each base may not be photolytically defined without a special photoresist process or other spatially-confined deblocking process. In another example, a donor substrate/wafer can be prepared with the AM1 synthesis and the RTP described above, but this time on a quartz substrate with cleavable silane, and all active bases can be added to the growing DNA sequence via the photoamidite method. The results of this experiment after gel transfer and hybridization with the fluorescently labelled complement can be similar to or substantially the same as those when using the DMT chemistry. The spatial resolution can be high, at approximately 3-4 μm line and space (L/S) pattern.

In some cases, to fully ensure that the process is compatible with all microarray fabrication requirements, a full 6 in wafer from Centrillion's pilot line (Palo Alto, Calif.) can be synthesized with the cleavable silane, UCL's and AM1 sequence using the photoamidite method. The hybridization results can be similar to or substantially the same as those when the AMT chemistry is used. A wafer scale transfer feasibility study can show that wafer scale transfer is feasible. The results can support that oligos can be transferred from a solid donor wafer to an acceptor wafer and be put onto an acrylamide gel using the above-described process on pieces of wafers of various sizes at relevant die geometries. It can be demonstrated that the ammonia diffusion through the polymerized sandwich is sufficient to support chemical cleavage of the moieties synthesized below the photo defined sequence, and that the chemistry utilized is compatible with the necessary phosphoramidite chemistry for microarray fabrication, as detected by complementary sequence hybridization.

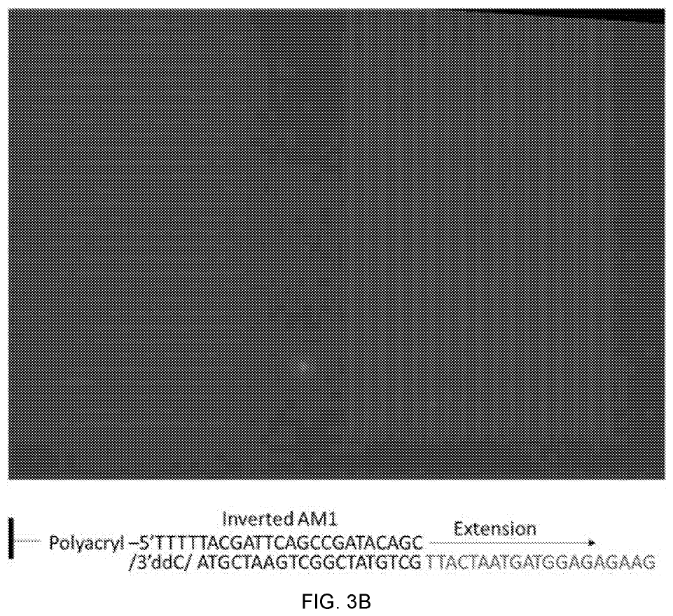

Since the transferred oligos are 3′-up with available hydroxyl groups, they can be responsive to various polymerase extension reactions. For example, the TAQ® Extension assay can be employed, on a portion of a 6 in wafer synthesized using the above-described methods. In this case, the quartz sacrificial wafer can have a single UCL and no cleavable silane to maintain compatibility with the control parts of the production wafer. A full 50-mer (5′ACGTTGGCTGACAGAGTGATCAGTGTCATAGTTGCGTTGGCAGGAATGTG3′, AM5) can be synthesized via photoamidite method, diced into individual smaller chips, and extended after gel transfer. All four bases can be present, but only the base T can be labelled with Cy3. FIG. 3A shows the results of the alignment markers after the above DNA synthesis, inversion onto gel, and extension reaction. The squares in the image are 3 μm, and interspersed with a second sequence that cannot extend. These results show that conversion of synthesized DNA can be detected in the 3′-up orientation. FIG. 3A shows fluorescent images of Cy3 labelled extended nucleotide from Taq polymerase-catalyzed extension reactions using labelled T only while in the presence of all 4 bases, 3 μm square features.

In order to further confirm the presence of inverted oligos, and to show that the 3′-up probes would be successful for a variety of polymerases, a Centrillion Hero2 extension assay was performed (FIG. 3B). In this case, all 4 bases may be labelled and be available for the enzyme-catalyzed extension reaction based on the hybridized template oligo (sequence shown in FIG. 3B), and the presence of dideoxy nucleotides ensuring only a single base addition for this experiment. FIG. 3B shows fluorescent images of Cy3 labelled extended nucleotide from Hero polymerase extension of labelled A in the presence of all 4 labelled bases. High “A” intensities and clear negative controls (no insertion of other bases, with the exception of small bleedover of C due to the filter sets) can demonstrate that the method is working as expected, and provide evidence for the availability of 3′ hydroxyl on the gel inverted oligos because no extension can be found with unmatched bases. These results of FIGS. 3A and 3B may demonstrate that the oligos originally synthesized as 5′-up orientation can be inverted to 3′-up orientation onto an acrylamide gel and become available for a variety of enzymatic reactions.

Recently, arrays have been proposed to use the nexus of array fabrication with commercial sequencing readouts. In these scenarios, and other potential applications, high resolution printing may be necessary. For example, arrays can be used to elucidate the positional information of biomolecules by attaching unique oligos patterned on arrays to sample of interest in situ; then analyzing the results using commercial sequencing readouts. In these scenarios, the spatial resolution of the biomolecules is naturally limited to the number of unique features that can be patterned into a given area. Therefore, sub-micron resolution of photolithographically patterned features can be of importance for array manufacturing. However, DMT chemistry is not directly compatible with photolithographically-based microarray probe synthesis, as each base cannot be photolytically defined without a special photoresist process or other spatially-confined deblock process.

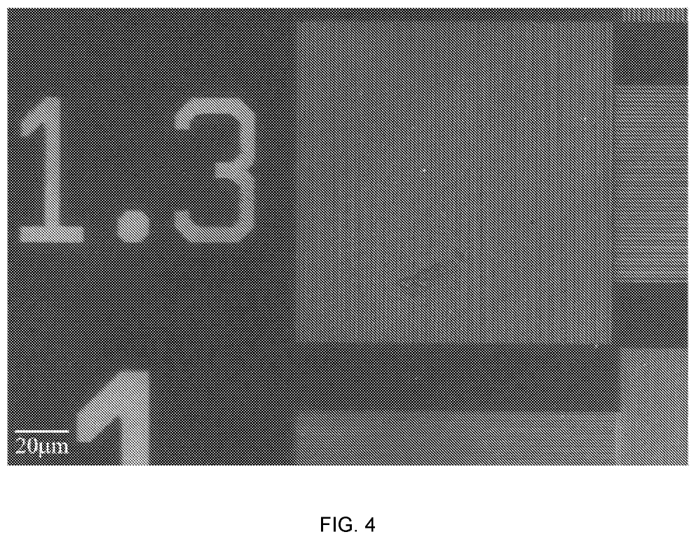

In order to test the resolution of the above-described gel inversion process, the Centrillion photoresist can be coated onto a second wafer with the AM1 probes as before, but this time on a quartz substrate with a cleavable silane, in order to demonstrate the high spatial resolution. All active bases were exposed via the photoamidite method. In this experiment, DMT chemistry can be used to synthesize a 20-mer sequence, and an in-line fluorescein label (6-FAM, Glen Research) can be added. The last T on the as-synthesized, 5′ end can be left with the DMT group on, and it can be imaged with the Centrillion photoresist which uses photoacid generator chemistry in a polymer matrix to spatially deblock the protecting group. Gel transfer can be performed as described earlier, with pieces of the donor substrates floated off and separated from the acceptor substrate in base solutions in about 18 hours. FIG. 4 shows the fluorescent image of the results of this experiment. The 1.0 μm line and space patterns can be resolved to the limit of the imaging tool (a microscope from Keyence, 40×, NA0.6), demonstrating that the lateral “blur” from the gel inversion process is likely or primarily associated with the molecular length of the oligos synthesized.

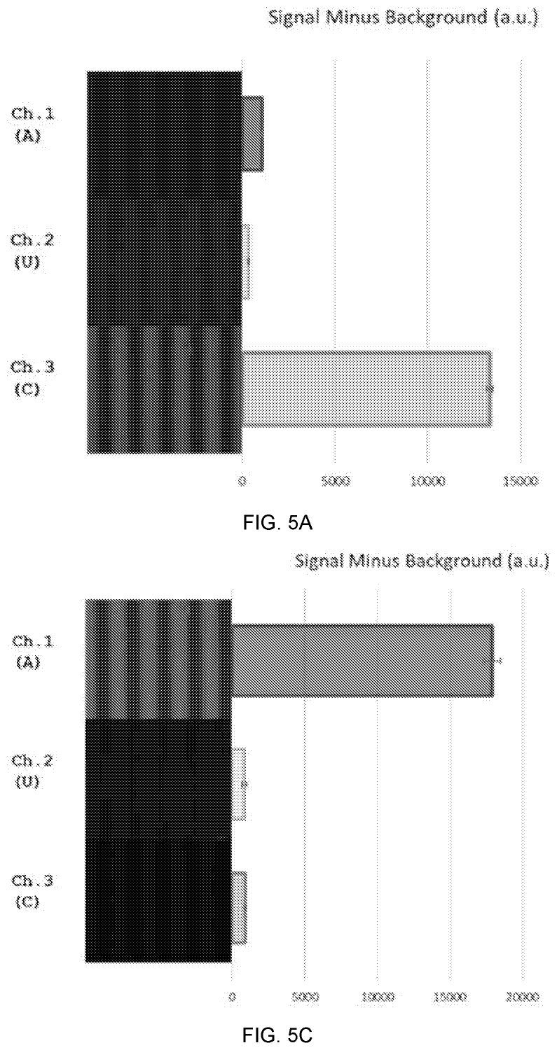

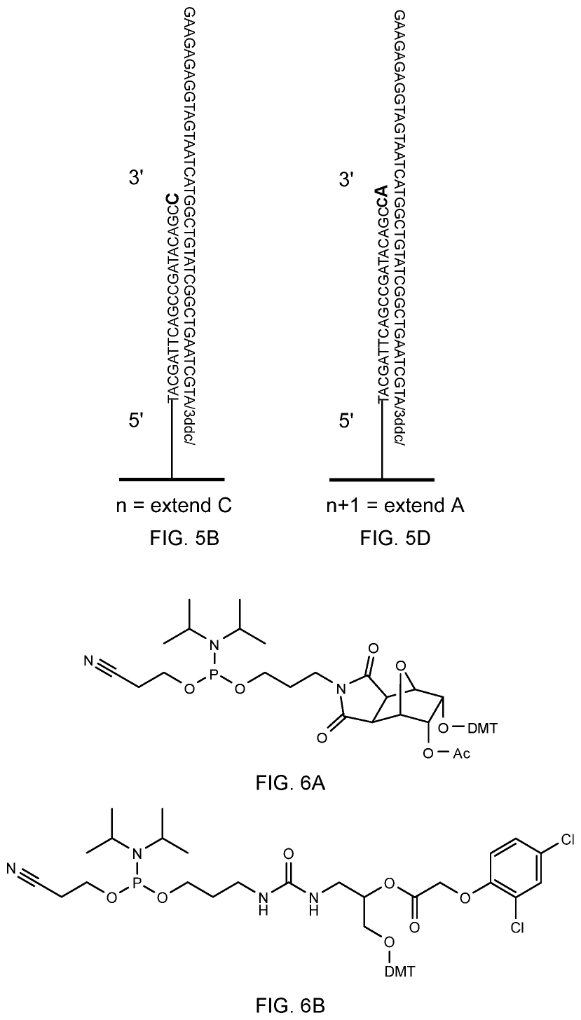

The 3′ up microarrays can be versatile tools for enzymatically-driven assays. As such, two polymerase-catalyzed reactions shown above (FIGS. 3A and 3B) can demonstrate that the 3′ hydroxyls are available for labelled base extension assays. Other enzymatic reactions can further demonstrate the utility of the 3′ up hydroxyl format using sequencing with reversible terminators on chip, and can demonstrate the versatility of the chips with respect to enzyme activity, selectivity, and future potential assay development. FIGS. 5A-5D show the results of a two-base extension using Centrillion's reversible terminator chemistry according to U.S. Patent Application No. 2016/0355541 A1 and International Patent Application No. WO 2016/182984, all of which are herein incorporated by reference for all purposes. In FIGS. 5A and 5B, the correct base (cytosine) can be added in the presence of the other labelled bases. Following cleavage of the label and the terminator (blocking group) on the 3′ hydroxyl, the second base can be added in a second round of extension with labelled reversible terminators (shown in FIGS. 5C and 5D). In the second round of extension, the correct incorporation of the second base (adenosine) can be shown in FIGS. 5C and 5D. The chips can be used for on-chip sequencing of nucleic acids.

Successful manufacture and transfer creating 3′-up oligos can yield oligos available for extension reactions catalyzed by polymerases, and the above results can demonstrate good extension efficiency and probe fidelity. To the limit of the detection method used, it may appear that no lateral displacement blur from the gel inversion process can be found as long as the sacrificial wafer (donor substrate) can be chemically cleaved from the product wafer (acceptor substrate).

It may be advantage t to have the ability to control the release of the bound oligos from the donor substrate after free radical polymerization with the gel on the acceptor substrate. Release prior to the gel formation may cause loss of probes and/or positional fidelity. In some cases, when physical removal of the donor wafer is attempted prior to the full chemical release from the acceptor substrate, high feature fidelity can be found at the edges, but poor fidelity and signal can be found in the center, indicating physical breaks can be occurring in the gel or perhaps part-way through the synthesized DNA. Conversely, full release of the cleavable moieties after the gel formation can provide good signal and feature fidelity across the chip/wafer. Chemical release after polymerization may bring about the substantial mass transfer problem of how to get chemical reagents to the interface for release.

Recognizing the problem related to the timing of the release of the donor substrate from the acceptor substrate, in some cases, laser perforation along the dicing streets may be introduced prior to the submersion of the “sandwich” in ammonia or other cleaving reagents. Not to be bound by any theories disclosed herein, the presence of the hydrogel in-between the substrates may cause the Fickian diffusion as one of the major mechanisms of getting the concentrated base for cleavage from the edge of the chip/wafer/die to the interior portion (e.g., the center) of the chip/wafer/die. At a D=1.64×10⁻⁵ cm²/s in water, the characteristic time for ammonia to reach the center of an about 1 cm die can be about 13 min. This may result in an ammonia solution reaching the interior or center of the substrates (i.e., the chip/wafer) at that point. Deprotections in a concentrated ammonia solution can be hours long, for example, about 1, 2, 3, 4, 5, 6, 7, 8, 9, 10, 11, 12, 13, 14, 15, 16, 17, 18, 19, or 20 hours long. In some cases, cleaving reaction to be completed in a short time, for example, in about 5, 10, 15, 20, 25, 30, 35, 40, 45, 50, 55, or 60 minutes with dilute ammonia or caustic solutions while maintaining high fidelity throughout the entire oligo synthesis cycle, can be achieved, for example, by using much higher activity releasing agents, such as AMA (1:1 mixture (v/v) of aqueous Ammonium hydroxide and aqueous methylamine). Altering temperature may also be an option in that the rate of cleavage will increase to shorten the waiting time for the cleavage.

It can be estimated that greater than 50% of the full length synthesized oligos can be transferred, based on comparison of fluorescent hybridization signals of inverted, 3-up oligos (on the acceptor wafer) versus as-synthesized 5′-up oligos (on the donor wafer). This can be consistent with the polymerization conditions chosen to react some, most, or all monomers during the polymerization process. Although an exact number of transferred oligos may not be known when using the hybridization method to confirm the presence of the 5′ up transferred oligos, even if the hybridization signals detected may range from 60-100% on the gel as compared to similarly processed, 5′-up oligos on the wafer. The hybridization method may not be precise because the plethora of short, capped sequences from the non-unity synthesis layer yield are not transferred to the gel as they do not get the acrylamide monomer moiety, hence lowering the overall charge field of the transferred oligos. Also, hybridization yields may be somewhat inversely proportional to surface oligo concentration in this range. Even if 50% of the oligos were transferred, it may be possible that one would detect similar signals in hybridization metrology due to increased hybridization efficiency. Nevertheless, since hybridization may be the initial step in many downstream assays, then the fact that the signals are as high as or higher than similarly synthesized 5′-up is an advantage for the present disclosure.

In summary, inversion of transferred oligos can be obtained using the methods described herein, as well as excellent results can be obtained from 3 different polymerase-catalyzed, extension assays on the inverted oligos. This inversion can be accomplished while simultaneously preserving the high spatial resolution (about 3 μm for photoamidite synthesis) requisite for microarray work, and even a demonstration of 1 μm lateral resolution required for potential readouts using commercial sequencers. This inversion method to fabricate DNA sequencing array is a powerful tool for extending the applicability, and providing for new applications of, DNA arrays and has the potential for enabling future applications such as DNA storage.

This new type of photolithographic DNA microarray where the array is patterned into a hydrogel with the oligos in the 3′ up configuration can have many advantages. For example, the array can have fewer sequencing errors and more oligos can be added to by polymerase, effectively permitting a wide range of substrate sequences to be programmed into the system for future applications development. The fabrication strategy can be compatible with existing machines and tools for synthesizing microarrays, relatively cheap to produce, and scalable to six-inch wafer processing. Positional fidelity of the array within the gel can be high, and the synthesis can be integrated with photoresist acid generator chemistry to produce features in the sub-micron range. Polymerase and restriction endonuclease assays can show that the patterned oligos can serve as substrates to different enzymes, and sequencing by synthesis demonstrates the utility of the array with more exotic substrates like fluorescent reversible terminators. This fabrication process can be a powerful tool for extending the applicability of DNA microarrays, potentially enabling applications such as genomic sequencing library construction via chip-based barcodes, and indexed DNA based data storage.

In some embodiments, the surface treatment of substrate can comprise binding oligothymidine groups covalently to the substrate. In some embodiments, the oligothymidine group thus attached to the surface can comprise 2, 3, 4, 5, 6, 7, 8, 9, 10, 11, 12, 13, 14, 15, or more thymidine nucleotides. In some embodiments, the oligothymidine group can comprise 5 thymidine nucleotides. In some embodiments, the free 5′ hydroxyl groups of the oligothymidine group can react with branched linker phosphoramidite and can be covalently attached thereto.

After the surface cleaning and treatment, reagents can react with the surface hydroxyl or amino groups. For example, the surface can react with a cleavable linker (CL) phosphoramidite through a reactive group, for example, a hydroxyl group. The cleavable linker (CL) phosphoramidite, including, for example, as a universal cleavable linker (UCL) phosphoramidite. Choices for the cleavable linker phosphoramidite can include, but are not limited to, molecules shown in FIG. 6. As used herein, the term cleavable linker, or CL (including UCL), generally refers to any of the following: a cleavable linker phosphoramidite reagent, a surfaced-bound cleavable linker before the addition of nucleotides, and a surfaced-bound branched linker after the addition of nucleotides. Cleavable linker phosphoramidite can react with the substrate using standard DNA synthesis protocols with some modifications, including, for example, adding the cleavable linker reagent to the DNA synthesis substrate, increasing the coupling time (e.g., 3 minutes), etc. In some embodiments, the cleavable linker phosphoramidite can react with free hydroxyl groups. In some embodiments, the cleavable linker can comprise a hydroxyl group protected by DMT. In some embodiments, the cleavable linker can comprise a primary hydroxyl group protected by DMT.

Then DNA sequence can be synthesized on the substrate according to standard DNA synthesizer protocols, with a capping step installed after each step of nucleic acid addition to block the unreacted free 3′ hydroxyls so that the truncated sequence would not continue with DNA chain elongation. Capping can be achieved by treatment with acetylation reagents.

After the final capping step, a reactive group bearing phosphoramidite can react with full-length DNA sequences, but not with truncated DNA sequencing on the 5′-end. The reactive group can immobilize with the gel in the sandwich format described earlier for the donor and acceptor substrate.

In some embodiments, the cleavable linker (or UCL) can be cleaved, for example, by reaction with NH₄OH, potassium carbonate, methyl amine, 1,2-diaminoethane (also known as ethylenediamine, EDA), potassium hydroxide in methanol, or AMA (a mixture of NH₄OH and methyl amine). Cleavage of the cleavable linker can release the 3′-OH terminus of all probe sequences, thereby releasing truncated probe sequences not immobilized to the gel.

In some embodiments, the cleavable linker can undergo cleavable under basic condition to cleave both full-length and truncated probe sequences from their 3′ end. Because of prior immobilization (or polymerization with the gel) provided a covalent bond between the 5′ end of the full-length probe sequences and the gel on the acceptor substrate, these probes can be inverted on the surface of the acceptor substrate to a 5′ to 3′ orientation. Meanwhile, the truncated probe sequences can be deleted from the acceptor surface and their only attachment to the donor substrate can be severed, thereby removing the truncated probe sequences from both substrates after washing. Consequently, in some embodiments, probe sequences left on the acceptor substrate can comprise mostly full-length probe sequences with 5′ to 3′ orientation. In some embodiment, the probe inversion step can increase the percentage of full-length probe sequences among all probe sequences when compared with the probes before the probe inversion step (i.e., on the donor substrate).

There may be several advantages of in situ probe inversion disclosed in the present disclosure. It may avoid the use of toxic reagents in certain chemical reactions. In addition, avoiding a separate cleavage step after DNA array synthesis may save time and reduce cost when applied at a larger scale. Removing a synthetic step may decrease operational mistakes which may occur during DNA array preparations.

UCL cleavage can occur when synthesized probes, both full-length probes and truncated probed, are treated with a base reagent, such as, for example, NH₄OH, ethylenediamine/water (EDA: water), or AMA (a mixture of NH₄OH and methyl amine). Because the full length probes can be immobilized onto the acceptor substrate, a free 3′-OH on the 3′ end of the full-length probe sequence with 5′ to 3′ orientation on the acceptor substrate can be obtained.

In one example, controlled pore glass (CPG) beads can be used as the synthesis substrate, which reacts with branched linker and cleavable linker. Then oligonucleotide probes can be synthesized on cleavable linkers attached to the substrate, including a reactive group at the 5′ end of the full-length probe sequence.

The probe inversion techniques discussed herein can be conducted in aqueous media. Avoidance of the use of organic solvents can make such techniques more environmentally friendly and increase the ease of chemical handling and waste disposal.

The probe inversion techniques discussed herein can be conducted at a pH of at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, or 13.5. The probe inversion techniques discussed herein can be conducted at a pH of at most about 14.0, 13.5, 13.0, 12.5, 12.0, 11.5, 11.0, 10.5, 10.0, 9.5, 9.0, 8.5, 8.0, 7.5, 7.0, 6.5, 6.0, 5.5, 5.0, 4.5, 4.0, 3.5, 3.0, 2.5, 2.0, 1.5, 1.0, or 0.5. The probe inversion techniques discussed herein can be conducted at a pH of about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, or 13.5. In some cases, the probe inversion techniques discussed herein can be conducted at or about physiological pH, such as about 7.365 or about 7.5. Conducting reactions at physiological pH can reduce or obviate the need for handling harsh substances or reaction conditions, and can employ aqueous media.

The probe inversion techniques discussed herein can be conducted at a temperature of about 15° C., 20° C., 25° C., 30° C., or 35° C. The probe inversion techniques discussed herein can be conducted at a temperature of at most about 15° C., 20° C., 25° C., 30° C., or 35° C. The probe inversion techniques discussed herein can be conducted at a temperature of at least about 15° C., 20° C., 25° C., 30° C., or 35° C. In some cases, the probe inversion techniques discussed herein can be conducted at or about room temperature, such as about 20° C., about 21° C., about 22° C., about 23° C., about 24° C., about 25° C., about 26° C., from about 20° C. to about 26° C., or from about 20° C. to about 22° C. Conducting reactions at room temperature can reduce or obviate the need for handling harsh substances or reaction conditions.

Releasing truncated probe sequences can increase the percentage of full-length sequences present in the array. In some cases, at least about 10%, 20%, 30%, 40%, 50%, 60%, 70%, 80%, 90%, 95%, 96%, 97%, 98%, 99%, 99.9%, 99.99%, or 99.999% of probes remaining bound to the array substrate following a probe inversion process are full-length sequences. In some cases, a probe inversion process can release at least about 10%, 20%, 30%, 40%, 50%, 60%, 70%, 80%, 90%, 95%, 96%, 97%, 98%, 99%, 99.9%, 99.99%, or 99.999% of truncated probes bound to the array substrate prior to the probe inversion process.

The synthesis substrate can comprise different forms or shapes, such as a bead or a flat array. The synthesis substrate can comprise any suitable material, including but not limited to glass (e.g., controlled pore glass), silicon, or plastic. Substrates can comprise polymer coatings or gels, such as a polyacrylamide gel or a PDMS gel. Gels and coatings can additionally comprise components to modify their physicochemical properties, for example, hydrophobicity. For example, a polyacrylamide gel or coating can comprise modified acrylamide monomers in its polymer structure such as ethoxylated acrylamide monomers, phosphorylcholine acrylamide monomers, betaine acrylamide monomers, and combinations thereof.

Inverted probes can provide many advantages over standard non-inverted probes, for a variety of applications. For example, as discussed above, probe inversion can remove most or all of undesired truncated probe sequences, thereby providing a population of inverted probes containing up to 100% full-length probe sequences. Additionally, inverted probes can have the 3′ OH group free, which can be beneficial for conducting enzymatic reactions (e.g., single or multiple base extension, ligase reaction, etc.). The inverted probes can also be used for sequencing by synthesis (SBS) process, among other applications.

### EXAMPLES

### Cleavable Silane Synthesis

2-Hydroxyethyl 3-(methyl(3-(trimethoxysilyl)propyl)amino)propanoate was synthesized as follows. N-Methyl-3-(trimethoxysilyl)propan-1-amine was cooled in an ice bath under nitrogen with stirring. 2-Hydroxyethylacrylate (HEA) was added dropwise while stirring over 30 min, and the reaction was left stirring under nitrogen for 24 hours at room temperature (RT), and stored undiluted.

### Chip and Gel Preparation

Substrates were cleaned in NANO-STRIP® (KMG), rinsed and exposed to a 3 wt % solution of a silanating reagent in 5% water in ethanol for 4 hours, washed, dried, and held in a desiccator for at least 24 hours before RT atmospheric storage and use. For donor wafers, the silanating reagent was the cleavable silane described above unless otherwise stated, and for the acceptor wafer, 3-acrylamidopropyltrimethoxysilane (Gelest). Silane coated, 2 in×3 in slides were placed into an ABI (Applied Biosystems) 394 synthesizer with a custom flow cell inserted in place of the column in the flow path. The flow cell was consisted of the substrate vacuum-held to an o-ring face seal. Reagents flowed into the cell as per normal DNA/RNA synthesis. Exposures on the ABI apparatus were done on the 2 in×3 in substrates and were performed with a custom exposure tool utilizing a 365 nm lamp housing with exposure through a proximity mask (Compugraphics, Fremont, Calif.). When specified, full 6 in wafers were prepared in a similar manner with a similarly-modified flow cell connected to a Dr. Oligo (Biolytic, Fremont, Calif.) synthesizer. Six in wafers were exposed on a Neutronix Quintel 8008AL (NxQ, Morgan Hill, Calif.) exposure tool in hard contact (vacuum between mask and substrate) in a cleanroom in Palo Alto, Calif., or when stated, a similar hard contact proximity exposure tool in Taiwan, ensuring intimate mask and wafer contact.

Photoamidites (i.e. photo-T) were utilized and exposed as described in literature. See McGall G. H., Christians F. C. (2002) High-Density GeneChip Oligonucleotide Probe Arrays. In: Hoheisel J. et al. (eds) Chip Technology. Advances in Biochemical Engineering/Biotechnology, vol 77, 21-42. Springer, Berlin, Heidelberg. Wafers had 5 dimethoxytrityl-blocked thymines (DMT-T's) placed on the bottom, or near or at the surface of the substrate and uniformly across the wafer prior to the addition of cleavable moieties of either one or two Universal Cleavable Linkers (UCL, AM Chemicals, P/N 02120, Oceanside, Calif.). Then the sequence of interest was synthesized in the direction of 3′->5′. After the sequence of interest was completed, another 4 DMT-T's were placed followed by patterning of the last T (either photo-T, or in the case of high-resolution demonstration, a DMT-T with photoresist) of interest followed by ACRYDITE™ (Glen Research, Sterling, Va.) addition. The 2 in×3 in substrates were then cleaved to ˜1 cm squares. Unless otherwise stated, a 5% tetramethylenediamine (TEMED, Aldrich, Milwaukee, Wis.) was prepared, a weighed 4.8 wt % solution of potassium persulfate (Aldrich), saturated, and a 5% acrylamide solution with 5% bi-functional group (Bio-Rad, Hercules, Calif., 161-0144) were prepared separately and outgassed for a minimum of 10 minutes and not exceeding 1 hour under nitrogen. About 200 μl of TEMED was added to 10 ml of the acrylamide solution. Then 250 μl of potassium persulfate (KPS) was added and quickly stirred all without exposure to the atmosphere. Approximately 20 μl of the reaction mixture was removed and added to the acrylamido-silane coated substrate in air and the patterned, sacrificial wafer pieces of either about 7.5 mm×about 7.5 mm die (diced from the 6 in wafers) or about 1 cm critical dimension rectangles diced from the synthesis wafer were inverted on top. No attempt was made to exclude oxygen at this point and polymerization presumably progressed after the free radicals overwhelmed the dissolved oxygen between the sandwiched wafers. As a result, the edges of the polymerized gel were rough as the oxygen-affected polymerization altered the gel properties.

The wafer “sandwich” with crosslinked pieces was placed in concentrated ammonia for 18 hours unless otherwise noted. For a full 6 in wafer gel transfer, approximately 300 μl of the polymerization mixture was applied to the as-synthesized, sacrificial 5′ up wafer and the quartz substrate put down on top, so the wicking of the polymerization mixture could be observed. In some cases, when the 2 in×3 in substrates were diced into 8-10 mm pieces and inverted, the sacrificial wafer pieces may float from the gel by the force of the solution movement from the orbital shaker. Where the full complement of release chemistries was not used, either because of compatibility concerns with manufacturing wafers, or early experiments where it wasn't yet clear the level of cleavable moieties required, gentle nudging may be required. The wafer sandwiches sat in the ammonia until release before a solution of ethylene diamine: water (50:50, Aldrich Milwaukee, Wis.) was applied for 1-3 hours to finish the deprotection and ensure the complete reduction of UCL to 3′ OH.

The gel wafers were then washed with water, followed by 4×SSC buffer (Aldrich) and were ready for hybridization. Hybridization was done with 25 nM of the complementary sequence, labelled at the 5′ end with Cy3 (IDT, Coralville, Iowa) overnight at 45° C. and allowed to cool for greater than 1 hour. The gel wafer was washed 3 times in 4×SSC, with the last held in wash solution for at least 5 min before imaging on a fluorescence microscope (Keyence BZ-X710 Itasca, Ill.).

### Probe Extension Assays

5′CTGTCTCTTATACACATCTGAGCTGAATTCATAACTTCGTATAGCATACATT ATACGAAGTTATGCTGTATCGGCTGAATCGTA, the 84 base template oligo, was ordered from IDT and hybridized to the inverted array for 2 hours in 2×SSC buffer at 45° C. The array was then washed in 1×SSC buffer for 15 minutes at RT, then two more times in 0.5×SSC for 15 minutes each at RT. Extension was done using the DNA polymerase Klenow Large Fragment (New England Biolabs, Ipswitch, Mass.) under standard conditions at 37° C. for 1 hour. The array was then washed in 1×SSC and submerged in a solution of 0.2 N NaOH for 10 minutes with shaking to strip away the template oligo, and finally equilibrated with 5 ml of 1×SSC. The Cy3 labeled probe targeting the Mosaic End sequence was then hybridized to the array and washed as before, then imaged on the Keyence BZ-X710.

### Patterning and Transfer Using the Centrillion Photoresist

To demonstrate high resolution, the AM1 oligo (5′TACGATTCAGCCGATACAGC3′) was prepared on 2 in×3 in substrate except that a 6-fluorescein phosphoramidite (6-FAM, Glen Research) was added in line, and the photo-T was replaced with DMT-T, and the DMT group left intact. The wafer was spin coated with the Centrillion Photoresist (Centrillion Technologies, Inc., Palo Alto, Calif.) at 2500 rpm for 1 min, baked in a convection oven at 50° C. for 5 min, exposed at 36 mJ/cm² and let sit at RT for 4 min. The resist was stripped in propylene glycol monomethyl ether acetate (PGMEA) and isopropanol. The substrate was blown dry with nitrogen and put back in the synthesizer for an Acrydite, inverted onto a gel, and imaged on the Keyence microscope using the FITC channel.

### On-Chip Stepwise Sequencing

The AM1 sequence (5′TACGATTCAGCCGATACAGC3′) was synthesized on chip with the ABI 394 DNA Synthesizer 5′ up with a patterned Acrydite and inverted onto the gel as before with a final wash in 8×SSC for 30 min RT. The sequence GAAGAGAGGTAGTAATCATGGCTCTATCGGCTGAATCGTA/3ddC/1 μM was hybridized in 8×SSC at 35° C., brought to RT in 30 min and washed. Extension occurred with all four bases present, 3 fluorescently labelled and bearing reversible terminators. The first base was added with fluorescent master mix (FLMM) and imaged in 3 channels to demonstrate correct base addition. The extension was completed with unlabeled reversible terminators, cleaved and imaged to verify loss of fluorescence. The process was then repeated with a second base with FLMM and imaged.

### Enzymatic Reactions With 3′ up Transferred Oligos

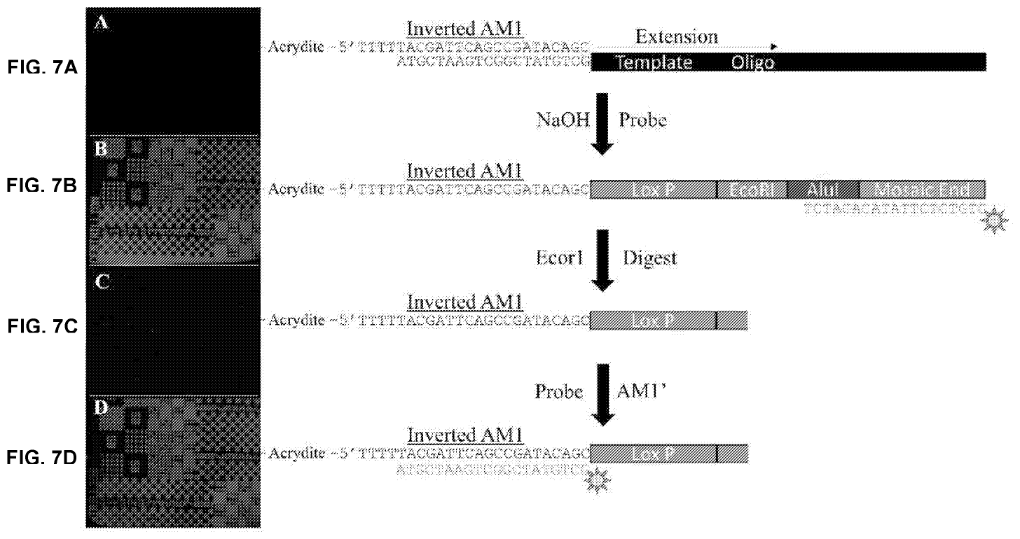

Since the transferred oligos are 3′-up with reactive hydroxyl groups, they can be responsive to polymerase extension reactions. To demonstrate that, 5′CTGTCTCTTATACACATCTGAGCTGAATTCATAACTTCGTATAGCATACATTATAC GAAGTT ATGCTGTATCGGCTGAATCGT, an 84 base template oligo containing the reverse complement of AM1 was hybridized to the array and extended with Klenow DNA polymerase (FIG. 7A). After the extension, the template oligo was stripped away with NaOH, the array was washed in SSC buffer, and finally the array was hybridized with a probe complementary to the last 20 bases on the 3′ ends of the newly extended molecules. FIG. 7B shows the results of the fluorescent probe hybridized to the newly synthesized region of the array. The resolution test pattern can be readily observed demonstrating efficient addition of 64 bases to the 3′ ends of the oligos on the array via enzyme-catalyzed extension reactions

The ability to copy long template DNA sequences onto the 3′ ends of densely patterned arrays is another advantage and unexpected results of the disclosed platform. Such an ability can allow molecular complexity to be added en masse to all features on an array simultaneously. As an example, the template oligo used in FIGS. 7A-7D was designed to encode: 1) the canonical LoxP sequence for Cre-mediated recombination between the array and any foxed DNA target; 2) the EcorI restriction sequence; 3) the AluI restriction sequence; and 4) the 19 base Mosaic End sequence recognized by Tn5 transposase. As polymerase reactions function in this system, the array can be constructed in a single or double stranded configuration. Both EcorI and AluI have been shown to cut single as well as double stranded DNA giving the researcher in this example the option of generating sticky or blunt array ends if desired. Meanwhile, Tn5 transposase has already been used to construct genomic DNA sequencing libraries on a hydrogel surface with Mosaic End oligos randomly dispersed in the gel. Given the length of the final molecules, photolithographically synthesizing an array with this many sequence motifs may not be achieved by using the standard phosphoramidite chemistry. In contrast, by using only 3′ up oligos from the transfer according to the present disclosure and then extending the 3′ oligos by polymerase, an error-free or substantially error-free microarray of the oligos can be generated.

To demonstrate that the inverted and extended array can function as a substrate for enzymes other than a polymerase, the array obtained from FIG. 7B (with the same fluorescent probe used in FIG. 7B still hybridized to the 3′ up oligos) was exposed to the restriction enzyme EcorI for 1 hour at 37° C. Then when imaged, the template pattern was virtually undetectable (shown in FIG. 3C), suggesting that the added enzyme had made an internal cut at the recognition sequence, liberating the 3′ Alu1 and Mosaic End sequences together with the hybridized fluorescent probe (FIG. 7C).

To ensure that cleavage was selective and not the result of nonspecific degradation of the array in the gel, a second Cy3 labelled probe was added and found to hybridize to the original AM1 sequence (FIG. 7D). As the resolution test pattern was again readily observed in FIG. 7D, one can conclude that digestion with EcorI is specific to the internal restriction sequence, leaving the Acrydite registered sequences 5′ of the cut intact.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of inverting an oligonucleotide on a surface, comprising:
(a) providing a donor substrate coupled with a plurality of molecules on a first surface of said donor substrate, a member of said plurality of molecules comprising (i) a first oligonucleotide in 3′ to 5′ orientation immobilized on said first surface of said donor substrate and (ii) a first reactive group attached to a 5′ end of said first oligonucleotide;
(b) providing an acceptor substrate comprising a plurality of second reactive groups immobilized on a surface of said acceptor substrate;
(c) arranging said donor substrate, a reaction mixture, and said acceptor substrate in a sandwich formation such that said first surface of said donor substrate is facing said surface of said acceptor substrate and said reaction mixture is placed in-between said first surface of said donor substrate and said surface of said acceptor substrate;
(d) subjecting said sandwich formation to an immobilization condition to form a first covalent bond between said first reactive group with said reaction mixture or derivative thereof, and a second covalent bond between a member of said plurality of second reactive groups and said reaction mixture or derivative thereof, thereby producing a transformed sandwich formation;
(e) releasing said donor substrate from said first oligonucleotide; and
(f) providing said first oligonucleotide in 5′ to 3′ orientation immobilized on said acceptor substrate via said reaction mixture or derivative thereof.

2. The method of claim 1, wherein in (f) said first oligonucleotide comprises a free 3′ hydroxyl group.

3. The method of claim 1, wherein said member of said plurality of molecules further comprises a universal cleavable linker in-between said first surface of said donor substrate and said first oligonucleotide in 3′ to 5′ orientation.

4. The method of claim 3, wherein said universal cleavable linker is coupled to said first surface via a reagent of

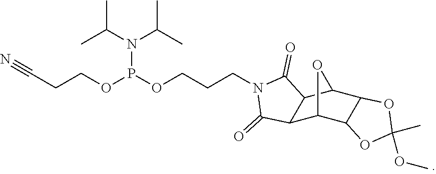 [SMILES: COC1(C)OC2C(O1)C1OC2C2C(=O)N(CCCOP(OCCC#N)N(C(C)C)C(C)C)C(=O)C12]

5. The method of claim 1, wherein said releasing in (e) comprises treating with a base.

6. The method of claim 5, wherein said base comprises at least one member selected from the group consisting of NH₄OH, 1,2-diaminoethane, and methyl amine.

7. The method of claim 1, wherein said immobilization condition is a polymerization reaction.

8. The method of claim 7, wherein said reaction mixture comprises a plurality of acrylamides for said polymerization reaction.

9. The method of claim 8, wherein said polymerization reaction forms a polymer gel, said polymer gel comprises said first covalent bond and said second covalent bond.

10. The method of claim 1, wherein said first reactive group comprises a first polymerizable group.

11. The method of claim 1, wherein said second reactive group comprises a second polymerizable group.

12. The method of claim 1, wherein in (a) said first oligonucleotide in 3′ to 5′ orientation is full-length.

13. The method of claim 12, wherein in (f) said first oligonucleotide in 5′ to 3′ orientation is full-length.

14. The method of claim 1, wherein in (e) said releasing further comprises performing a mechanical dicing process or a laser perforation process on a second surface of said donor substrate.

15. The method of claim 14, wherein in (e) subsequent to said performing said mechanical dicing process or said laser perforation process, said releasing further comprises treating with a base.

16. The method of claim 1, wherein said plurality of molecules form a pattern on said first surface of said donor substrate.

17. The method of claim 16, wherein in (f) said providing comprises converting said plurality of molecules in to a plurality of inverted molecules on said surface of said acceptor substrate, and wherein said plurality of inverted molecules keep said pattern on said surface of said acceptor substrate.

18. A method of preparing an oligonucleotide array in 5′ to 3′ orientation immobilized on an acceptor surface of an acceptor substrate, comprising:
(a) providing a sandwich formation, said sandwich formation comprising:
(i) a donor substrate comprising a donor surface;
(ii) a plurality of oligonucleotides, a 3′ end of each member of said plurality of oligonucleotides being covalently bonded to said donor surface;
(iii) a middle layer covalently bonded to a 5′ end of said member of said plurality of oligonucleotides; and
(iv) an acceptor substrate comprising an acceptor surface, said middle layer being covalently bonded to said acceptor surface;
(b) removing said donor substrate from said plurality of said plurality of oligonucleotide; and
(c) providing said oligonucleotide array in 5′ to 3′ orientation on said acceptor surface of said acceptor substrate.

19. The method of claim 18, further comprising: prior to (a), forming said middle layer from a mixture of reagents in between said donor surface bonding with said plurality of oligonucleotides and said acceptor surface.

20. The method of claim 19, wherein said forming said middle layer comprises conducting a polymerization reaction.

21. The method of claim 20, wherein said polymerization reaction polymerizes acrylamide reagents.

22. The method of claim 18, wherein said 3′ end of said member of said plurality of oligonucleotides is covalently bonded to a universal cleavable linker at said 3′ end of said member, said universal cleavable linker being covalently bonded to said donor surface.

23. The method of claim 22, wherein said removing in (b) comprises breaking a bond between said universal cleavable linker and said member of said plurality of oligonucleotides.

24. The method of claim 23, wherein said removing in (b) further comprises performing a mechanical dicing process or a laser perforation process on another surface of said donor substrate before said breaking said bond.

25. The method of claim 23, wherein said breaking said bond comprises treating said universal cleavable linker with a basic reagent.

26. The method of claim 23, wherein said basic reagent comprises at least one member selected from the group consisting of NH₄OH, 1,2-diaminoethane, and methyl amine.

27. The method of claim 18, wherein after (b) said middle layer remains covalently bonded to said acceptor surface.

28. The method of claim 27, wherein after (c) said oligonucleotide array remains in 5′ to 3′ orientation and covalently bonded to said middle layer via said 5′ end of said member of said plurality of oligonucleotides.

29. The method of claim 27, wherein after (c) each member of said oligonucleotide array comprises a free 3′ hydroxyl group.

30. The method of claim 19, wherein prior to said forming said middle layer, synthesizing said plurality of oligonucleotides from said donor surface in said 3′ to 5′ orientation.

31. A composition comprising:
(a) a donor substrate comprising a donor surface;
(b) a plurality of oligonucleotides, each member of said plurality of oligonucleotides being covalently bonded to said donor surface at a 3′ end of said member of said plurality of oligonucleotides;
(c) a middle layer covalently bonded to a 5′ end of said member of said plurality of oligonucleotides; and
(d) an acceptor substrate comprising an acceptor surface, said middle layer being covalently bonded to said acceptor surface.

32. The composition of claim 31, wherein said member of said plurality of oligonucleotides is covalently bonded to a universal cleavable linker via said 3′ end of said member of said plurality of oligonucleotides.

33. The composition of claim 32, wherein said universal cleavable linker is covalently bonded to said donor surface.

34. The composition of claim 31, wherein said donor substrate is configured to be mechanically diced or laser perforated into multiple pieces.

35. The composition of claim 31, wherein said middle layer comprises polyacrylamide.

36. The composition of claim 31, wherein said donor substrate is a Silicon wafer.

37. The composition of claim 31, wherein said acceptor substrate is a quartz wafer.

38. The composition of claim 31, wherein each member of said plurality of oligonucleotides comprises a free 3′ hydroxyl.

39. The composition of claim 31, wherein said composition is characterized in a combination of any two or more selected from the group consisting of:
(i) said member of said plurality of oligonucleotides is covalently bonded to a universal cleavable linker via said 3′ end of said member of said plurality of oligonucleotides;
(ii) said donor substrate is configured to be mechanically diced or laser perforated into multiple pieces;
(iii) said middle layer comprises polyacrylamide;
(iv) said donor substrate is a Silicon wafer;
(v) said acceptor substrate is a quartz wafer; and
(vi) each member of said plurality of oligonucleotides comprises a free 3′ hydroxyl.

40. A composition comprising:
(a) a substrate comprising a surface;
(b) a middle layer comprising a first surface and a second surface, said first surface being proximal to said surface of said substrate and said second surface being distal to said surface of said substrate, said first surface covalently bonded to said surface of said substrate; and
(c) a plurality of oligonucleotides covalently bonded to said second surface of said middle layer via 5′ ends of said plurality of oligonucleotides.

41. The composition of claim 40, wherein said 5′ ends of said plurality of oligonucleotides bonded to said second surface via carbon-carbon bonds.

42. The composition of claim 40, wherein said substrate is quartz.

43. The composition of claim 40, wherein said middle layer comprises polyacrylamide.

44. The composition of claim 40, wherein said surface of said substrate is bonded to said first surface via carbon-carbon-bonds.

45. The composition of claim 40, wherein each member of said plurality of oligonucleotides comprises a free 3′ hydroxyl.

46. The composition of claim 40, wherein said composition is characterized in a combination of any two or more selected from the group consisting of:
(i) said 5′ ends of said plurality of oligonucleotides bonded to said second surface via carbon-carbon bonds;
(ii) said substrate is quartz;
(iii) said middle layer comprises polyacrylamide;
(iv) said surface of said substrate is bonded to said first surface via carbon-carbon-bonds; and
(v) each member of said plurality of oligonucleotides comprises a free 3′ hydroxyl.

47. The composition of any one of claims 18-46, wherein said middle layer is about 10 μm, 15 μm, 20 μm, 25 μm, or 30 μm thick.
